(19) United States
(12) Patent Application Publication
(10) Pub. No.: US 2003/0046732 A1
(43) Pub. Date: Mar. 6, 2003

(54) Methods for regulating plant GABA production

(76) Inventors: Alan M. Kinnersley, East Lansing, MI (US); Frank J. Turano, Baltimore, MD (US)

Correspondence Address: Gregory B. Coy, Woodard, Emhardt, Naughton, Moriarty and McNett, Bank One Center/Tower, 111 Monument Circle, Suite 3700, Indianapolis, IN, 46204-5137, US

(21) Appl. No.: 10/006,852

(22) Filed: Nov. 7, 2001

Related U.S. Application Data
(60) Provisional application No. 60/246,367, filed on Nov. 7, 2000.

(51) Int. Cl. A01H 5/00

(52) U.S. Cl. 800/288; 435/468; 800/289

(57) ABSTRACT

The present invention relates to methods and compositions for regulating plant GABA production. More particularly, the invention relates to the use of polynucleotides that encode functional plant GAD enzymes for enhancing a plant's ability to produce. In various aspects, the invention provides methods of treating plants, vectors and other nucleic acid molecules useful for the treatments, and transformed plants better able to tolerate environmental or other plant stress.

### REFERENCES TO RELATED APPLICATIONS

This application claims the benefit of U.S. Provisional Application No. 60/246,367, filed Nov. 7, 2000, entitled METHODS FOR REGULATING PLANT GABA PRODUCTION, which is hereby incorporated by reference herein in its entirety.

### BACKGROUND OF THE INVENTION

The present invention relates to methods and materials for plant GABA production. Plants having an enhanced ability to produce GABA, and having desirable morphological and/or agronomic characteristics, environmental stress resistance, or the like, are provided through plant genetic engineering. More particularly, the invention relates to genetic transformation of plants with genes that enhance a plant's ability to produce GABA, thereby enhancing the plant's ability withstand stress or imparting other desirable characteristics, by encoding proteins that catalyze the conversion of glutamic acid to GABA.

As a background to the invention, the enzyme GAD (glutamic acid decarboxylase) has been shown to catalyze the formation of γ-aminobutyric acid (GABA) from glutamate (Glu), and in the last decade several plant GAD genes have been cloned. It has recently been reported that plant GADs have 22-25 additional amino acids at the C-termini when compared to the deduced amino acid sequences of GADs from other kingdoms and that these amino acids constitute a calmodulin-binding domain (CaM-BD). These domains have been shown to be sufficient for the binding of calmodulin (CaM) in the presence of Ca²⁺. Recently, the present inventors have demonstrated that two recombinant GAD (RGAD) isoforms, RGADI and rGAD2, from Arabidopsis did bind to CaM in the presence of Ca²⁺ and that the Ca²⁺/CaM complex stimulates GAD activity.

The CaM-BD functions as an autoinhibitory domain to deactivate the GAD enzyme. Uninhibited GAD activity, via the removal of the CaM-BD, has been shown to result in morphological, biochemical, and reproductive changes in transgenic tobacco plants. The genetically engineered tobacco plants that constitutively expressed a GAD gene minus the CaM-BD were stunted, sterile, and contained high levels of GABA and low Glu when compared with control plants.

The rapid accumulation of GABA in plant cells after exposure to stress has been well documented. In plants, there are at least three metabolic pathways that give rise to GABA. The first pathway is associated with the catabolism of polyamines. The second pathway is part of the GABA shunt, the reversible GABA aminotransferase reaction. The third pathway is via the decarboxylation of glutamate. The latter reaction is proposed to be the major source of GABA accumulation in plants after stress. Furthermore, the rapid accumulation of GABA has been observed with an increase of in vitro GAD activity.

Results from several experiments have demonstrated that radioactively labeled ¹⁴C-Glu is synthesized into ¹⁴C-GABA. Asparagus cells incubated in ¹⁴C-Glu for ten minutes rapidly produce GABA. However it may be argued that the production of GABA in that system is a non-physiological response to cells in suspension culture. Likewise, detached developing soybean cotyledons injected with ¹⁴C-Glu produce ¹⁴C-GABA. Like the experiment mentioned above, the result could be a nonphysiological response of the detached cotyledon. However, this work suggests that GABA is the normal route for Glu metabolism in developing soybean cotyledons and that GABA biosynthesis is not a response to stress under these circumstances.

The results from the ¹⁴C-Glu experiments demonstrate that Glu is converted to GABA, via GAD in isolated plant cells and detached organs. GABA has been shown to rapidly accumulate in plants subjected to mechanical stimulation, cold shock and heat shock conditions that have been shown to elevate cytosolic Ca²⁺ concentrations. In view of this background, it is seen that significant effort has been devoted to studying GABA synthesis and GAD enzyme activity in plants; however, a direct role for GABA in plants has not heretofore been demonstrated. The present invention is a significant advance in this field.

### SUMMARY OF THE INVENTION

The present invention relates to methods and compositions for regulating plant GABA production. More particularly, the invention relates to the use of polynucleotides that encode functional plant GAD enzymes. In various aspects, the invention provides methods for transforming plants, vectors and other nucleic acid molecules useful therein, and transformed plants have the advantage of enhanced GABA production, such as, for example, enhanced ability to tolerate environmental or other plant stress.

In one aspect of the invention, polynucleotides encoding functional plant GAD enzymes are used to transform cells and to transform plants. Inventive methods produce plants that have advantages of enhanced GABA production, such as, for example having enhanced plant growth characteristics, survival characteristics and/or tolerance to environmental or other plant stresses, without causing stunting or other deleterious morphological alterations. Plants are genetically modified in accordance with the invention to introduce into the plant a polynucleotide encoding a GAD enzyme that functions in the formation of increased amounts of GABA in the plant. The polynucleotide is operably linked at its 5′ end to a promoter sequence that controls, or otherwise regulates, transcription of the polynucleotide.

In certain forms of the invention, modified polynucleotides are provided that encode a constitutively activated, and otherwise deregulated GAD enzyme lacking an autoinhibitory calmodulin binding domain. Polynucleotides encoding wild type, regulatable GAD that includes an autoinhibitory calmodulin binding domain are used in other forms of the invention. Overproduction of deregulated or other GAD (such as wild type GAD) results in increased synthesis of GABA in the plant. Increased concentrations of GABA are beneficial to the plant, by, for example, decreasing the deleterious effects of plant stress.

It is an object of the present invention to provide methods of treating plants, vectors and other nucleic acid molecules useful for the treatments, and transformed plants that feature modified GABA production.

Further objects, advantages and features of the present invention will be apparent from the detailed description herein.

### BRIEF DESCRIPTION OF THE FIGURES

Although the characteristic features of this invention will be particularly pointed out in the claims, the invention itself, and the manner in which it may be made and used, may be better understood by referring to the following description taken in connection with the accompanying figures forming a part hereof.

FIG. 1 depicts a graph showing the effect of mechanical stimulation on accumulation of GABA in wild-type Arabidopsis as more fully described in Example Two.

FIG. 2 depicts an immunoblot analysis of wild type and antiGAD2 plants as more fully described in Example Three. The proteins were blotted to nitrocellulose, stained for protein to confirm equal loading (B), destained, and GAD2 peptide was detected (A) by immunoblot analysis.

FIG. 3 depicts images of wild-type and antiGAD2 plants twenty-four hours after heat shock treatment as described more fully in Example Four.

FIG. 4 depicts the results of the mechanical stimulation procedures described more fully in Example Five.

FIG. 4A depicts a statistical comparison of bolt height, and

FIG. 4B is an image of plants as described in Example Six.

FIG. 5 depicts an immunoblot analysis of wild type, rGAD2 and trunGAD2 plants as more fully described in Example Six. The proteins were blotted to nitrocellulose, stained for protein to confirm equal loading (B), destained, and GAD peptides were detected (A) by immunoblot analysis.

### DETAILED DESCRIPTION OF THE INVENTION

For purposes of promoting an understanding of the principles of the invention, reference will now be made to particular embodiments of the invention and specific language will be used to describe the same. It will nevertheless be understood that no limitation of the scope of the invention is thereby intended, such alterations and further modifications in the invention, and such further applications of the principles of the invention as described herein being contemplated as would normally occur to one skilled in the art to which the invention pertains.

The present invention relates to methods and compositions for regulating plant GABA production. The invention specifically relates to transformed plants that feature enhanced production of GABA, and advantages associated therewith, such as, for example, being better able to tolerate environmental or other plant stress and/or having enhanced agronomic characteristics. The invention also relates to DNA constructs, vectors and other nucleic acid molecules and methods for making transformed plants.

In accordance with the invention, plants are genetically modified by introducing into a plant host cell a polynucleotide encoding a functional plant GAD enzyme, operably linked at its 5′ end to a promoter that controls, or otherwise regulates, transcription of the polynucleotide. As used herein, “plant GAD enzyme” refers to a glutamic acid decarboxylase enzyme that functions in a plant to convert glutamic acid to γ-aminobutyric acid (GABA). Plant GAD enzymes are well known to persons of ordinary skill in the art, and examples include polypeptides having the amino acid sequences set forth in SEQ ID NOS: 2, 4, 6, 8, 10, 12, 14, 16 and 18. SEQ ID NOS: 2, 4, 6, 8 and 10 set forth Arabidopsis thaliana GAD1, GAD2, GAD3, GAD4 and GAD5, respectively; SEQ ID NOS: 12 and 14 set forth Tobacco NtGAD1 and NtGAD2, respectively; SEQ ID NO: 16 sets forth Petunia GAD; and SEQ ID NO: 18 sets forth Tomato GAD.

It has recently been reported that plant GAD enzymes contain a calmodulin-binding domain (CaM-BD) consisting of 22-25 additional amino acids at the C-termini that are not present in the deduced amino acid sequences of GADs from other kingdoms. These domains have been shown to be sufficient for the binding of calmodulin (CaM) in the presence of Ca²⁺. It has now been reported that the CaM-BD functions as an autoinhibitory domain to deactivate the GAD enzyme. Recently, the present inventors have demonstrated that two recombinant GAD (rGAD) isoforms, rGAD1 and rGAD2, from Arabidopsis did bind to CaM in the presence of Ca²⁺ (Turano, F. J. and Fang, T. K. (1998) Characterization of two glutamate decarboxylase cDNA clones from Arabidopsis thaliana. Plant Physiol. 117: 1411-1421) and that the Ca²⁺/CaM complex stimulates GAD activity.

Removal of the CaM-BD has been shown to result in a GAD enzyme exhibiting uninhibited GAD activity, resulting in uninhibited GABA production. The term “plant GAD enzyme” also encompasses GAD peptides lacking the calmodulin binding domain. Indeed, in certain forms of the invention, the uninhibited production of GABA upon expression of the GAD enzyme is an advantageous and desirable feature.

It is also envisioned in accordance with the invention that the calmodulin binding domain of GAD can be modified in other ways (i.e., other than being completely removed), which modifications result in elimination of the autoinhibitory function of the calmodulin binding domain. Such modified GAD enzymes are expressly contemplated by the present invention.

With respect to descriptions herein relating to a “polynucleotide encoding a plant GAD enzyme,” the term “polynucleotide,” refers to a natural or synthetic linear and sequential array of nucleotides and/or nucleosides, including deoxyribonucleic acid, ribonucleic acid, and derivatives thereof. The terms “encoding” and “coding” refer to the process by which a polynucleotide, through the mechanisms of transcription and translation, provides the information to a cell from which a series of amino acids can be assembled into a specific amino acid sequence to produce a functional polypeptide, such as, for example, an active enzyme or other protein that has a specific function.

A suitable polynucleotide for use in accordance with the invention may be obtained by cloning techniques using cDNA or genomic libraries of Arabidopsis thaliana which are available commercially or which may be constructed using standard methods known in the art. Suitable nucleotide sequences may be isolated from DNA libraries obtained from a wide variety of species by means of nucleic acid hybridization or polymerase chain reaction (PCR) procedures, using as probes or primers nucleotide sequences selected in accordance with the invention, such as those set forth in SEQ ID NOS: 1, 3, 5, 7, 9, 11, 13, 15 and 17, other polynucleotides described herein, or portions thereof. In preferred forms of the invention, the polynucleotides provided herein are cDNA sequences.

Alternately, a suitable sequence may be made by techniques that are well known in the art. For example, polynucleotides encoding a plant protein described herein may be constructed by recombinant DNA technology, for example, by cutting or splicing nucleic acids using restriction enzymes and DNA ligase. Furthermore, nucleic acid sequences may be constructed using chemical synthesis, such as solid-phase phosphoramidate technology, or PCR. PCR may also be used to increase the quantity of nucleic acid produced. Moreover, if the particular nucleic acid sequence is of a length which makes chemical synthesis of the entire length impractical, the sequence may be broken up into smaller segments which may be synthesized and ligated together to form the entire desired sequence by methods known in the art.

As stated above, an inventive DNA construct includes a promoter that directs transcription in a plant cell, operably linked to the polynucleotide encoding a plant GAD enzyme. In various aspects of the invention described herein, a variety of different types of promoters are described and used. As used herein, a polynucleotide is “operably linked” to a promoter or other nucleotide sequence when it is placed into a functional relationship with the promoter or other nucleotide sequence. The functional relationship between a promoter and a desired polynucleotide insert typically involves the polynucleotide and the promoter sequences being contiguous such that transcription of the polynucleotide sequence will be facilitated. Two nucleic acid sequences are further said to be operably linked if the nature of the linkage between the two sequences does not (1) result in the introduction of a frame-shift mutation; (2) interfere with the ability of the promoter region sequence to direct the transcription of the desired nucleotide sequence, or (3) interfere with the ability of the desired nucleotide sequence to be transcribed by the promoter sequence region. Typically, the promoter element is generally upstream (i.e., at the 5′ end) of the nucleic acid insert coding sequence.

While a promoter sequence can be ligated to a coding sequence prior to insertion into a vector, in other embodiments, a vector is selected that includes a promoter operable in the host cell into which the vector is to be inserted (that is, the a promoter that is recognized by the RNA polymerase of the host cell). In addition, certain preferred vectors have a region which codes for a ribosome binding site positioned between the promoter and the site at which the DNA sequence is inserted so as to be operatively associated with the DNA sequence of the invention once inserted (in correct translational reading frame therewith). The vector should be selected to provide a region that codes for a ribosomal binding site recognized by the ribosomes of the host cell into which the vector is to be inserted. A “plant promoter” is a promoter capable of initiating transcription in plant cells.

A wide variety of promoters are known in the art, as are other regulatory elements that can be used alone or in combination with promoters, and a wide variety of promoters that direct transcription in plants cells can be used in connection with the present invention. For purposes of describing the present invention, promoters are divided into two types, namely, constitutive promoters and non-constitutive promoters, including, for example, tissue preferred promoters, tissue specific promoters, cell specific promoters and inducible promoters.

Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in certain tissues are referred to as “tissue-specific.” A “cell type” specific promoter primarily drives expression in certain cell types in one or more organs, for example, vascular cells in roots or leaves. An “inducible” promoter is a promoter that is under environmental control. Examples of environmental conditions that may effect transcription by inducible promoters include mechanical shock, heat, cold, salt, flooding, drought, wounding, anoxia, pathogens, ultraviolet-B, nutritional deprivation and combinations thereof. Tissue-specific, tissue-preferred, cell type specific, and inducible promoters constitute the class of “non-constitutive” promoters. A “constitutive” promoter is a promoter that is active under most environmental conditions, such as, for example, CaMV 35S promoter and the nopaline synthase terminator.

Of particular interest in certain embodiments of the present invention are inducible promoters that respond to various forms of environmental stresses, or other stimuli, including, for example, mechanical shock, heat, cold, salt, flooding, drought, salt, anoxia, pathogens, such as bacteria, fungi, and viruses, and nutritional deprivation, including deprivation during times of flowering and/or fruiting, and other forms of plant stress. For example, the promoter selected in alternate forms of the invention, can be a promoter induced by abiotic stresses such as wounding, cold, dessication, ultraviolet-B [van Der Krol et al. (1999) Plant Physiol. 121:1153-1162], heat shock [Shinmyo et al., (1998) Biotechnol. Bioeng. 58:329-332] or other heat stress, drought stress or water stress. The promoter may further be one induced by biotic stresses including pathogen stress, such as stress induced by a virus [Sohal et al. (1999) Plant Mol. Biol. 41:75-87] or fungi [Eulgem (1999) EMBO. J. 18:4689-4699], stresses induced as part of the plant defense pathway [Lebel (1998) Plant J. 16:223-233] or by other environmental signals, such as light [Ngai et al. (1997) Plant J. 12:1021-1034; Sohal et al. (1999) Plant Mol. Biol. 41:75-87], carbon dioxide [Kucho et al. (1999) Plant Physiol 121:1329-1338], hormones or other signaling molecules such as auxin, hydrogen peroxide and salicylic acid [Chen and Singh (1999) Plant J. 19:667-677], sugars and gibberellin [Lu et al. (1998) J. Biol. Chem. 273:10120-10131] or abscissic acid and ethylene [Leubner-Metzger et al. (1998) Plant Mol. Biol. 38:785-795].

In other embodiments of the invention, tissue specific promoters are used. Examples of tissue specific expression patterns as controlled by tissue or stage-specific promoters include fiber specific, green tissue specific, root specific, stem specific, and flower specific. For the protection of plants against foliar pathogens, expression in leaves is preferred; for the protection of plants against flower and fruit pathogens, expression in inflorescences (e.g. spikes, panicles, cobs etc.) is preferred; for protection of plants against root pathogens, expression in roots is preferred; for protection of seedlings against soil-borne pathogens, expression in roots and/or seedlings is preferred. In many cases, however, protection against more than one type of phytopathogen will be sought, and thus expression in multiple tissues will be desirable.

Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the plant GAD genes in a transformed plant or cell. Promoters suitable for expression in green tissue include many which regulate genes involved in photosynthesis and many of these have been cloned from both monocotyledons and dicotyledons. A suitable promoter is the maize PEPC promoter from the phosphenol carboxylase gene (Hudspeth et al. 1989. Plant Molec. Biol. 12: 579-589). A suitable promoter for root specific expression is that described by de Framond (1991. FEBS 290: 103-106) or by Hudspeth et al. (1996. Plant Molec. Biol. 31: 701-705). A suitable stem specific promoter is that described in patent application WO 93/07278 (to Ciba-Geigy) and which drives expression of the maize trpA gene.

The promoters may further be selected such that they require activation by other elements known in the art, so that production of the protein encoded by the nucleic acid sequence insert may be regulated as desired.

A promoter selected for use in an inventive construct can be an endogenous promoter, i.e. a promoter native to the species and or cell type being transformed. Alternatively, the promoter can be a foreign promoter. A “foreign promoter” is defined herein to mean a promoter, other than the native, or natural, promoter, which promotes transcription of a length of DNA of viral, bacterial or eukaryotic origin, including those from plants and plant viruses. For example, in certain preferred embodiments, the promoter may be of viral origin, including a cauliflower mosaic virus promoter (CaMV), such as CaMV 35S or 19S, a figwort mosaic virus promoter (FMV 35S), or the coat protein promoter of tobacco mosaic virus (TMV). The promoter may further be, for example, a promoter for the small subunit of ribulose-1,3-diphosphate carboxylase. Promoters of bacterial origin include the octopine synthase promoter, the nopaline synthase promoter and other promoters derived from native Ti plasmids as discussed in Herrera-Estrella et al., Nature, 303:209-213 (1983).

In addition to the selection of a suitable promoter, DNA constructs for plant GAD protein expression in plants require an appropriate transcription terminator to be attached downstream of the plant GAD gene. Several such terminators are available and known in the art (e.g. tml from CaMV, E9 from rbcS). A wide variety of available terminators known to function in plants can be used in the context of this invention.

In one form of the invention, a DNA construct comprising a non-constitutive promoter operably linked to a polynucleotide encoding a functional plant GAD enzyme is used to make a transformed plant that selectively increases production of GABA in response to a signal. As used herein, the term “signal” is used to refer to a condition, stress or stimulus that results in or causes a non-constitutive promoter to direct expression of a coding sequence operably linked thereto. To make such a plant in accordance with the invention, a DNA construct is provided that includes a non-constitutive promoter operably linked to a polynucleotide encoding a functional plant GAD enzyme. The construct is incorporated into a plant's genome to provide a transformed plant that expresses the polynucleotide in response to a signal. In alternate embodiments of the invention, the selected promoter is a tissue preferred promoter, a tissue specific promoter, a cell type specific promoter, an inducible promoter or other type of non-constitutive promoter.

It is readily apparent that such a DNA construct causes a plant transformed thereby to selectively express the GAD enzyme, or to increase expression of GAD, under specific conditions or in certain tissues or cell types. The result of this expression vis-à-vis GABA production in the plant depends upon the activity of the encoded GAD enzyme and in some cases the conditions of the cell or cells in which it is expressed. In one embodiment, the polynucleotide is a truncated polynucleotide that encodes a GAD enzyme lacking an autoinhibitory calmodulin binding domain. Thus, the truncated GAD enzyme expressed is constitutively activated, or deregulated. It is, of course, understood that, although the enzyme encoded in this embodiment is constitutively activated, the non-constitutive promoter does not continuously produce the truncated GAD enzyme. Rather, the promoter selected for inclusion in the promoter advantageously induces or increases transcription of the truncated GAD polynucleotide in a plant in response to a signal, such as, for example, in the presence of environmental or other plant stress, including biotic and/or abiotic stresses, or other conditions.

Polynucleotides encoding wild type, regulatable GAD that includes the autoinhibitory calmodulin binding domain are utilized in other embodiments of the invention. It will be understood by a person of ordinary skill in the art that in embodiments including a non-constitutive promoter and a GAD enzyme including a calmodulin binding domain, two conditions will result in production of increased amounts of GABA compared to a non-transformed plant. In particular, increased GABA production in a plant transformed with such a construct is conditioned first upon occurrence of a signal to which the selected promoter responds, which results in increased expression of GAD. In addition, activity of the GAD enzyme expressed is conditioned upon occurrence of conditions effective to activate the GAD enzyme. As is readily understood by a person of ordinary skill in the art, this condition can be met by the coexistence of calmodulin and calcium ions in proximity to the GAD enzyme, or by occurrence of other conditions. A plant transformed with such a construct advantageously exhibits heightened GABA production under the conditions discussed, and the benefits thereof, such as, for example, an enhanced ability to withstand a stress.

In another form of the invention, a DNA construct comprising a constitutive promoter operably linked to a polynucleotide encoding a functional plant GAD enzyme is used to make a transformed plant that constitutively increases production of GABA in a transformed plant. To make such a plant in accordance with the invention, a DNA construct is provided that includes a constitutive promoter operably linked to a polynucleotide encoding a functional plant GAD enzyme. The construct is incorporated into a plant's genome to provide a transformed plant that expresses the polynucleotide.

It is readily understood by a person of ordinary skill in the art that such a DNA construct causes a plant transformed thereby to constitutively express the GAD enzyme, the result of which, vis-à-vis GABA production in the plant, depends upon the activity of the encoded GAD enzyme and in some cases the conditions of the cell or cells in which it is expressed. In one embodiment, the polynucleotide is a truncated polynucleotide that encodes a GAD enzyme lacking an autoinhibitory calmodulin binding domain. Thus, the truncated GAD enzyme expressed is constitutively activated, or deregulated. Because the constitutive promoter directs constitutive expression of the GAD enzyme, and the enzyme encoded in this embodiment is constitutively activated, a plant transformed with such a construct exhibits an overall increase in GABA content.

Although it has been reported that excessive overproduction of GABA in a plant can result in stunting and other undesirable agronomic and/or morphological characteristics, the present invention recognizes that non-excessive overproduction of GABA in a plant results in beneficial characteristics, such as, for example, enhanced stress resistance or other desirable morphological and/or agronomic characteristics. Thus, the invention provides, after transformation of one or more plants, selecting a transformed plant exhibiting a desired level of GABA production by selecting a transformed plant having one or more desired morphological and/or agronomic characteristic, or by rejecting a transformed plant exhibiting undesirable stunting, sterility, loss of yield, loss of plant height, or other undesirable characteristic. In one embodiment, the desired characteristic selected for is the character of non-sterility. In another embodiment, the plant selected is not significantly stunted compared to a non-transformed plant under corresponding conditions. In another embodiment, a plant is selected based upon a retention of suitable yield characteristics compared to a non-transformed plant.

In another embodiment, a plant is selected based upon a GABA concentration in non-stress conditions of up to about 0.28 milligrams GABA per grams dry weight (mg/GDW). In yet another embodiment, a plant is selected based upon a GABA concentration in non-stress conditions of up to about 0.24 milligrams GABA per grams dry weight (mg/GDW). In still another embodiment, a plant is selected based upon a GABA concentration in non-stress conditions of up to about 0.20 milligrams GABA per grams dry weight (mg/GDW). In still another embodiment, a plant is selected based upon a GABA concentration in non-stress conditions of from about 0.10 about 0.28 milligrams GABA per grams dry weight (mg/GDW). In still another embodiment, a plant is selected based upon a GABA concentration in non-stress conditions of from about 0.10 about 0.24 milligrams GABA per grams dry weight (mg/GDW). In still another embodiment, a plant is selected based upon a GABA concentration in non-stress conditions of from about 0.10 about 0.20 milligrams GABA per grams dry weight (mg/GDW).

Polynucleotides encoding wild type, regulatable GAD that includes the autoinhibitory calmodulin binding domain are utilized with a constitutive promoter in other embodiments of the invention. It will be understood by a person of ordinary skill in the art that in embodiments including a constitutive promoter and a GAD enzyme including a calmodulin binding domain, a transformed plant constitutively expresses the GAD enzyme, but it is believed that the enzyme itself remains in a substantially inhibited conformation until occurrence of conditions effective to activate the GAD enzyme. As is readily understood by a person of ordinary skill in the art, this condition can be met by the coexistence of calmodulin and calcium ions in proximity to the GAD enzyme or by other conditions.

Overproduction of deregulated or other GAD (such as wild type GAD) provides for increased synthesis of GABA, increased levels of which are beneficial to the plant, by, for example, decreasing the effects of plant stress. The introduced polynucleotide, in an appropriate vector, is advantageously integrated into the plant genome, but may remain episomal in other forms of the invention.

A wide variety of vectors may be employed to transform a plant, plant cell or other cell with a construct made or selected in accordance with the invention, including plasmids (including high and low copy number plasmids), phage vectors (including λZap and pBluescript) and cosmids. Such vectors, as well as other vectors, are well known in the art. Representative T-DNA vector systems are discussed in the following publications: An et al., (1986) EMBO J. 4:277; Herrera-Estrella et al., (1983) EMBO J. 2:987; Herrera-Estrella et al., (1985) in Plant Genetic Engineering, New York: Cambridge University Press, p. 63. The vectors can be chosen such that the GAD gene, operably linked to a promoter as described herein, will become incorporated into the genome of the plant.

In one embodiment, the desired recombinant vector may be constructed by ligating DNA linker sequences to the 5′ and 3′ ends of the desired nucleotide insert, cleaving the insert with a restriction enzyme that specifically recognizes sequences present in the linker sequences and the desired vector, cleaving the vector with the same restriction enzyme, mixing the cleaved vector with the cleaved insert and using DNA ligase to incorporate the insert into the vector as known in the art.

The vectors may include other polynucleotides, such as those encoding selectable markers, including those for antibiotic resistance or color selection. The vectors may further include other regulatory elements, such as enhancer sequences, which cooperate with the promoter to achieve transcription of the nucleic acid insert coding sequence. By “enhancer” is meant nucleotide sequence elements that can stimulate promoter activity in a cell, such as a plant host cell. The vectors may further include 3′ regulatory sequence elements known in the art, such as those, for example, that increase the stability of the RNA transcribed.

Moreover, the vectors may include another polynucleotide insert that encodes a peptide or polypeptide used as a tag to aid in purification of the desired protein encoded by the desired nucleotide sequence. The additional polynucleotide is positioned in the vector such that a fusion, or chimeric, protein is obtained. For example, a protein described herein may be produced having at its C-terminal end linker amino acids, as known in the art, joined to the other protein that acts as a tag. After purification procedures known to the skilled artisan, the additional amino acid sequence is cleaved with an appropriate enzyme. The protein may then be isolated from the other proteins, or fragments thereof, by methods known in the art. In another embodiment, a vector includes another polynucleotide that encodes a plant GABA receptor protein, as described in the inventors' copending U.S. patent application Ser. No. 09/517,438. Alternatively, plants can be transformed in accordance with the invention with two different vectors, one including a DNA construct for expression of a GAD enzyme, and the other for expression of a plant GABA receptor protein or other polypeptide. It is expected that overexpression of a GAD enzyme and a GABA receptor protein in a plant will result in a plant with excellent features, such as, for example, enhanced stress resistance.

With respect to the use of inventive recombinant vectors to transform a host plant or cell, inventive methods include introducing into a plant cell a nucleic acid having a nucleotide sequence as described herein. Methods of transforming a plant are well known in the art, and may be found in references including, for example, Maniatis et al., Molecular Cloning: A Laboratory Manual, Cold Springs Laboratory, Cold Springs Harbor, N.Y. (1982) and Current Protocols in Molecular Biology, John Wiley and Sons, edited by Ausubel et al. (1988). Plant gene transfer techniques may also be found in references including Fromm et al., (1985) Proc. Natl. Acad. Sci. USA, 82:5824-5828 (lipofection); Crossway et al., (1986) Mol. Gen. Genet. 202:179 (microinjection); Hooykaas-Van Slogtem et al., (1984) Nature 311:763-764)(T-DNA mediated transformation of monocots); Rogers et al., (1986) Methods Enzymol. 118:627-641 (T-DNA mediated transformation of dicots); Bevan et al., (1982) Ann. Rev. Genet. 16:357-384) (T-DNA mediated transformation of dicots); Klein et al., (1988) Proc. Natl. Acad. Sci USA 85:4305-4309 (microprojectile bombardment); and Fromm et al., Nature (1986) 319:791-793 (electroporation). Once the desired nucleic acid has been introduced into the host cell, the host cell expresses the protein, or variants thereof, as described above. Accordingly, in yet another aspect of the invention, a host cell is provided that includes the inventive recombinant DNA constructs described above.

A wide variety of host cells may be used in the invention, including prokaryotic and eukaryotic host cells. Preferred host cells are eukaryotic and are further preferably plant cells, such as, for example, those derived from monocotyledons, such as duckweed, corn, turf (including rye grass, Bermuda grass, Blue grass, Fescue), dicotyledons, including lettuce, cereals such as wheat, crucifers (such as rapeseed, radishes and cabbage), solanaceae (including green peppers, potatoes and tomatoes), and legumes such as soybeans and bush beans.

The polynucleotides may be introduced into a plant utilizing standard techniques of molecular biology as found, for example, in Current Protocols in Molecular Biology, John Wiley and Sons, edited by Ausubel et al. (1988) and Maniatis, et al., Molecular Cloning, A Laboratory Manual, Cold Spring Harbor Laboratory (1989). For example, promoter sequences or polynucleotides described herein can first be incorporated into a vector and the vector can be introduced into the cell by a wide variety of techniques known to the art, including, for example, electroporation methods, lipofection methods, Agrobacterium-mediated gene transfer techniques, microinjection techniques, and microprojectile bombardment. Many gene transfer techniques, as well as others, may be found in, for example, the Ausubel et al. and Maniatis, et al. publications referenced above, as well as in Fromm et al., (1985) Proc. Natl. Acad. Sci. USA, 82:5824-5828 (lipofection); Crossway et al., (1986) Mol. Gen. Genet. 202:179 (microinjection); Hooykaas-Van Slogtem et al., (1984) Nature 311:763-764)(T-DNA mediated transformation of monocots); Rogers et al., (1986) Methods Enzymol. 118:627-641 (T-DNA mediated transformation of dicots); Bevan et al., (1982) Ann. Rev. Genet. 16:357-384) (T-DNA mediated transformation of dicots); Klein et al., (1988) Proc. Natl. Acad. Sci USA 85:4305-4309 (microprojectile bombardment); and Fromm et al., Nature (1986) 319:791-793 (electroporation). Moreover, the polynucleotides may be incorporated into single or multiple vectors.

In one embodiment of the invention, a transformed host cell may be cultured as known in the art to produce a transformed plant. In this regard, a transformed plant can be made, for example, by transforming a cell, tissue or organ from a host plant with an inventive DNA construct; selecting a transformed cell, cell callus, somatic embryo, or seed which contains the DNA construct; regenerating a whole plant from the selected transformed cell, cell callus, somatic embryo, or seed; and selecting a regenerated whole plant that expresses the polynucleotide.

Transformed plants produced herein have the ability to enzymatically produce GABA constitutively or under selected conditions. Thus, a transformed plant includes a polynucleotide encoding a functional plant GAD enzyme that converts glutamic acid to GABA, including a GAD enzyme with or without a functional calmodulin binding domain. Other polynucleotides encoding enzymes that function to produce GABA are also contemplated by the invention.

The methods described above may be applied to transform a wide variety of plants, including decorative or recreational plants or crops, but are particularly useful for treating commercial crops. Examples of plants, and especially crops, that may be transformed to form transformed plants in the present invention, include monocotyledons, such as duckweed, corn, turf (including rye grass, Bermuda grass, Blue grass, Fescue), dicotyledons, including lettuce, cereals such as wheat, crucifers (such as rapeseed, radishes and cabbage), solanaceae (including green peppers, potatoes and tomatoes), and legumes such as soybeans and bush beans. Further included in the invention are crops harvested from such plants and foodstuff containing them. In one embodiment of the invention, a plant transformed in accordance with the invention is selected from the group consisting of duckweed, rice, wheat, barley, rye, corn, Bermuda grass, Blue grass, fescue, rapeseed, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, bush beans, tobacco, tomato, green pepper, sorghum and sugarcane.

Once transformed, the plant may be treated with other “active agents” either prior to or during the exposure of the plant to stress to further decrease the effects of plant stress. “Active agent”, as used herein, refers to an agent that has a beneficial effect on the plant or increases production of GABA by the plant. For example, the agent may have a beneficial effect on the plant with respect to nutrition, and the resistance against, or reduction of, the effects of plant stress.

Accordingly, the active agent may include a wide variety of fertilizers, pesticides and herbicides known in the art. Suitable fertilizers are disclosed, for example, in Kirk-Othmer, Concise Encyclopedia of Chemical Technology, 4th Ed. v. 10, pp. 433-514(1993). Other greening agents fall within the definition of “active agent” as well, including minerals such as magnesium and iron. The pesticides protect the plant from pests or disease and may be either chemical or biological and include fungicides, bactericides, insecticides and anti-viral agents as known in the art.

Although reference is made herein to exemplary plant GAD enzymes having amino acid sequences as set forth in the Sequence Listing appended hereto, and forms thereof that lack a calmodulin binding domain, it is understood that the invention is not limited to these specific amino acid sequences. Skilled artisans will recognize that, through the process of mutation and/or evolution, polypeptides of different lengths and having differing constituents, e.g., with amino acid insertions, substitutions, deletions, and the like, may arise that are related to, or sufficiently similar to, a sequence set forth herein by virtue of amino acid sequence homology and advantageous functionality as described herein. Also included within the scope of the invention, are variants of the polypeptides that function to catalyze the conversion of glutamic acid to GABA, as described herein.

It is well known that plants of a wide variety of species commonly express and utilize homologous proteins, which include the insertions, substitutions and/or deletions discussed above, and yet which effectively provide similar function. For example, an amino acid sequence isolated from other species may differ to a certain degree from the sequences set forth in SEQ ID NOS: 2, 4, 6, 8, 10, 12, 14, 16 and 18, and yet have similar functionality with respect to catalytic and regulatory function. Amino acid sequences comprising such variations are included within the scope of the present invention and are considered substantially or sufficiently similar to a reference amino acid sequence. Although it is not intended that the present invention be limited by any theory by which it achieves its advantageous result, it is believed that the identity between amino acid sequences that is necessary to maintain proper functionality is related to maintenance of the tertiary structure of the polypeptide such that specific interactive sequences will be properly located and will have the desired activity, and it is contemplated that a polypeptide including these interactive sequences in proper spatial context will have good activity, even where alterations exist in other portions thereof.

In this regard, a variant of the proteins described herein is expected to be functionally similar to those set forth in SEQ ID NOS: 2, 4, 6, 8, 10, 12, 14, 16 and 18, for example, if it includes amino acids which are conserved among a variety of plant species or if it includes non-conserved amino acids which exist at a given location in another plant species that expresses the proteins described herein.

Another manner in which similarity may exist between two amino acid sequences is where a given amino acid of one group (such as a non-polar amino acid, an uncharged polar amino acid, a charged polar acidic amino acid or a charged polar basic amino acid) is substituted with another amino acid from the same amino acid group. For example, it is known that the uncharged polar amino acid serine may commonly be substituted with the uncharged polar amino acid threonine in a polypeptide without substantially altering the functionality of the polypeptide. Whether a given substitution will affect the functionality of the enzyme may be determined without undue experimentation using synthetic techniques and screening assays known in the art.

In one embodiment of the invention, a polynucleotide selected for use in an inventive DNA construct encodes a functional plant GAD comprising an amino acid sequence having at least about 60% identity to an amino acid sequences set forth herein and is effective to catalyze conversion of glutamic acid to GABA. In another embodiment, the construct includes a polynucleotide encoding a functional GAD comprising an amino acid sequences having at least about 70% identity to an amino acid sequence set forth herein. In yet another embodiment, the construct includes a polynucleotide encoding a functional GAD comprising an amino acid sequences having at least about 80% identity to an amino acid sequence set forth herein. In still another embodiment, the construct includes a polynucleotide encoding a functional GAD comprising an amino acid sequences having at least about 90% identity to an amino acid sequence set forth herein.

Percent identity may be determined, for example, by comparing sequence information using the MacVector computer program, version 6.0.1, available from Oxford Molecular Group, Inc. (Beaverton, Oreg.). Briefly, the MacVector program defines identity as the number of identical aligned symbols (i.e., nucleotides or amino acids), divided by the total number of symbols in the shorter of the two sequences. The program may be used to determine percent identity over the entire length of the proteins being compared. Preferred default parameters for the MacVector program include: for pairwise alignment: (1) matrix=BLOSUM30; (2) Alignment speed−fast; (3) Ktuple=1; (4) Gap penalty=1; Top diagonals=5; Window size=5; for multiple alignment: matrix=BLOSUM series, open gap penalty=10; extended gap penalty=0.1, delay divergent=40%; protein gap parameters: Gap separation distance=8; residue-specific penalties=yes or on; hydrophilic residues=GPSNDQEKR.

The Sequence Listing also sets forth nine nucleotide sequences, identified as SEQ ID NOS: 1, 3, 5, 7, 9, 11, 13, 15 and 17, that encode the amino acid sequences set forth in SEQ ID NOS: 2, 4, 6, 8, 10, 12, 14, 16 and 18. It is also understood that the invention contemplates alternative polynucleotides that differ from the nucleotide sequences specifically set forth herein, but that encode a functional plant GAD enzyme. In particular, the invention expressly contemplates in alternate embodiments a DNA construct including a polynucleotide that encodes a protein having an amino acid sequence within the identity parameters specified above.

The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

It is therefore understood that the invention encompasses more than the specific exemplary polynucleotides encoding the proteins described herein. For example, modifications to a sequence, such as deletions, insertions, or substitutions in the sequence, which produce “silent” changes that do not substantially affect the functional properties of the resulting polypeptide molecule are expressly contemplated by the present invention. For example, it is understood that alterations in a nucleotide sequence which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

Nucleotide changes which result in alteration of the N-terminal and C-terminal portions of the encoded polypeptide molecule would also not generally be expected to alter the activity of the polypeptide. In some cases, it may in fact be desirable to make mutations in the sequence in order to study the effect of alteration on the biological activity of the polypeptide. Each of the proposed modifications is well within the routine skill in the art.

In one embodiment, the polynucleotide selected for use in a DNA construct in accordance with the invention has a sequence that encodes a functional plant GAD enzyme. In another embodiment, the polynucleotide has a sequence that encodes a functional plant GAD enzyme, and has a sequence sufficiently similar to the coding region of a reference polynucleotide that it will hybridize therewith under moderately stringent conditions. This method of determining similarity is well known in the art to which the invention pertains. Briefly, moderately stringent conditions are defined in Sambrook et al., Molecular Cloning: A Laboratory Manual, 2nd ed. Vol. 1, pp. 101-104, Cold Spring Harbor Laboratory Press (1989) as including the use of a prewashing solution of 5× SSC (a sodium chloride/sodium citrate solution), 0.5% sodium dodecyl sulfate (SDS), 1.0 mM ethylene diaminetetraacetic acid (EDTA) (pH 8.0) and hybridization and washing conditions of 55° C., 5× SSC.

In yet another embodiment, a polynucleotide is selected that encodes a functional plant GAD enzyme, and has at least about 70 percent identity to the coding region of a nucleotide sequence set forth in SEQ ID NO: 1,3, 5, 7, 9, 11, 13, 15 or 17. In another embodiment, a polynucleotide is selected that encodes a functional plant GAD enzyme, and has at least about 80 percent identity to the coding region of a nucleotide sequence set forth in SEQ ID NO: 1,3, 5, 7, 9, 11, 13, 15 or 17. In another embodiment, a polynucleotide is selected that encodes a functional plant GAD enzyme, and has at least about 90 percent identity to a specified length within the coding region of a nucleotide sequence set forth in SEQ ID NO: 1,3, 5, 7, 9, 11, 13, 15 or 17. In alternate embodiments, the specified length is about 100, about 200, about 300, about 800 or about 900 nucleotides, or the entire coding sequence. The percent identity may be determined, for example, by comparing sequence information using the MacVector program, as described above with reference to amino acid identity. Preferred default parameters include: (1) for pairwise alignment parameters: (a) Ktuple=1; (b) Gap penalty=1; (c) Window size=4; and (2) for multiple alignment parameters: (a) Open gap penalty=10; (b) Extended gap penalty=5; (c) Delay divergent=40%; and (d) transitions=weighted.

In another aspect, the invention contemplates the use of nucleotide sequences described herein for other purposes. For example, in certain cases, it is desirable to suppress expression of a cell's or a plant's native GAD enzyme. A non-limiting example includes a situation in which it is desirable to suppress expression of native GAD genes having calmodulin binding domains while at the same time selectively directing expression of a GAD enzyme lacking a calmodulin binding domain, and thereby designing an expression system that produces GABA only in response to one or more specifically selected signals.

Accordingly, this invention also provides strategies for manipulating a gene involved in GABA production and thus is an invaluable tool for further research of cellular stress and/or developmental processes. For example, manipulation of a plant GAD gene can provide quantitative information on the role of GABA-related processes on metabolic fluxes, nutrient utilization and storage, cellular differentiation, growth, senescence, and signaling. Such manipulation also provides a method for increasing crop productivity through enhancing crop resistance to biotic and abiotic stresses. Crop quality and yield is improved by increasing tolerance to a variety of environmental stresses, including disease. Both stress and disease cause a decrease in photosynthetic and nitrogen efficiency of crop plants resulting in decreased yields.

Gaining control of this process through the use of gene promoters or over-expression, activation-tagging (Weigel D, Ahn J H, Blazquez M A, Borevitz J O, Christensen S K, Fankhauser C, Ferrandiz C, Kardailsky I, Malancharuvil E J, Neff M M, Nguyen J T, Sato S, Wang Z Y, Xia Y, Dixon R A, Harrison M J, Lamb C J, Yanofsky M F, Chory J. 2000. Activation tagging in arabidopsis. Plant Physiol 2000 122:1003-1014), co-suppression(Elmayan T, Balzergue S, Beon F, Bourdon V, Daubremet J, Guenet Y, Mourrain P, Palauqui J C, Vernhettes S, Vialle T, Wostrikoff K, Vaucheret H 1998. Arabidopsis mutants impaired in cosuppression. Plant Cell 10:1747-1758) (Palauqui J C and Vaucheret H, Transgenes are dispensable for the RNA degradation step of cosuppression. 1998. Proc Natl Acad Sci USA 95:9675-9680), “knockout” (Azpiroz-Leehan, R. and Feldmann, K. A. (1997) T-DNA insertion mutagenesis in Arabidopsis: going back and forth. Trends Genet 13:152-156) (Krysan, P. J., Young, J. C. and Sussman, M. R. (1999) T-DNA as an insertional mutagen in Arabidopsis. Plant Cell 11:2283-2290) (Meissner, R. C., Jin, H., Cominelli, E., Denekamp, M., Fuertes, A., Greco, R., Kranz, H. D., Penfield, S., Petroni, K., Urzainqui, A., Martin, C., Paz-Ares, J., Smeekens, S., Tonelli, C., Weisshaar, B., Baumann, E., Klimyuk, V., Marillonnet, S., Patel, K., Speulman, E., Tissier, A. F., Bouchez, D., Jones, J. J. D., Pereira, A., Wisman, E. and Bevan, M. (1999) Function Search in a Large Transcription Factor Gene Family in Arabidopsis: Assessing the Potential of Reverse Genetics to Identify Insertional Mutations in R2R3 MYB Genes. Plant Cell 11: 1827-1840), or antisense constructs of the GAD homologs provides an opportunity (1) for inhibiting GABA production through “silencing” the gene, thereby altering, by lowering, the plant stress (biotic or abiotic) signal and altering valuable agronomic traits such as increased size or productivity and (2) for selectively triggering GABA production utilizing methods of over-expressing the plant GAD gene(s), leading to signaling, for example, and thus halting the further spread of a pathogen or environmental damage through plant tissues or cellular damage via an increased response to stress.

With respect to antisense suppression, the invention provides a method that includes introducing into a plant cell an antisense polynucleotide having a nucleotide sequence complementary to a nucleotide sequence provided herein, preferably a coding region thereof, such as one that is complementary to a nucleotide sequence set forth in SEQ ID NO: 1, 3, 5, 7, 9, 11, 13, 15 or 17 or a nucleotide sequence having at least about 70% identity, more preferably at least about 80% identity, most preferably at least about 90% identity to a length of nucleotides therein. The antisense nucleotide may have a length of about 20 to about 400 nucleotides, about 20 to about 800 nucleotides, about 20 to about 1400 nucleotides or about 20 to about 1800 nucleotides. In another embodiment, the antisense polynucleotide is as long as the entire length of the coding region of a nucleotide sequence set forth in SEQ ID NO: 1, 3, 5, 7, 9, 11, 13, 15 or 17.

The antisense polynucleotide may hybridize to the template strand, which serves as the strand from which RNA is produced, so that transcription will be reduced. Alternatively, the antisense polynucleotide may be complementary to, and therefore hybridize to, the RNA sequence, such as the mRNA sequence, transcribed from the nucleotide sequences described herein, so that translation of the mRNA sequence to express the encoded protein, such as a GAD enzyme, will be reduced. The antisense polynucleotide may be either DNA or RNA, and may include nucleotides that are linked by phosphodiester bonds. The antisense polynucleotide may also be modified as known in the art for increased stability. For example, the antisense polynucleotide may include nucleotides that are linked by phosphorothioate bonds, or may include modified bases as known in the art. Such antisense oligonucleotides may be purchased commercially, or may be synthesized utilizing methods known to the art, including use of automated synthesizers.

Preferred antisense oligonucleotides are complementary to the coding region of a particular polynucleotide, although the sequences may in addition bind to selected sequences in a non-coding region. In further preferred forms of the invention, the antisense oligonucleotides will bind to nucleotides adjacent to the ATG initiation codon.

As will be appreciated by a person of ordinary skill in the art upon consideration of the descriptions herein, one form of the present invention is a method for making a transformed plant that selectively increases production of GABA in response to a signal. This method includes incorporating into a plant's genome a DNA construct comprising a non-constitutive promoter operably linked to a polynucleotide that encodes a functional plant GAD enzyme, to provide a transformed plant; wherein the transformed plant expresses the polynucleotide in response to a signal. In certain embodiments, the promoter is selected from the group consisting of a tissue preferred promoter, a tissue specific promoter, a cell type specific promoter and an inducible promoter. In other embodiments, the promoter is an inducible promoter that is responsive to a signal selected from the group consisting of mechanical shock, heat, cold, salt, flooding, drought, wounding, anoxia, pathogens, ultraviolet-B, nutritional deprivation, a flowering signal, a fruiting signal, cell specialization and combinations thereof. In one embodiment, the GAD enzyme is a modified GAD that does not include a functional autoinhibitory calmodulin-binding domain.

A wide variety of target plants are contemplated in accordance with the invention. In one embodiment, the target plant is selected from the group consisting of duckweed, rice, wheat, barley, rye, corn, Bermuda grass, Blue grass, fescue, rapeseed, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, bush beans, tobacco, tomato, green pepper, sorghum and sugarcane.

Transformation of a plant can be accomplished in a wide variety of manners within the purview of a person of ordinary skill in the art. In one embodiment, a DNA construct is incorporated into a plant by (i) transforming a cell, tissue or organ from a host plant with the DNA construct; (ii) selecting a transformed cell, cell callus, somatic embryo, or seed which contains the DNA construct; (iii) regenerating a whole plant from the selected transformed cell, cell callus, somatic embryo, or seed; and (iv) selecting a regenerated whole plant that expresses the polynucleotide. The invention also provides transformed plants obtained according to the invention and progeny thereof, including a transformed plant in which the DNA construct is incorporated into the plant in a homozygous state.

In another form of the invention, there is provided a DNA construct comprising a non-constitutive promoter operably linked to a polynucleotide that encodes a GAD enzyme; wherein the promoter regulates expression of the polynucleotide in a host cell in response to a signal. In one embodiment, the promoter is a tissue specific plant promoter. In another embodiment, the promoter is an inducible plant promoter. The invention also provides a vector useful for transforming a cell, the vector comprising the DNA construct as described above. In another aspect, the invention, provides a cell having incorporated therein a foreign gene comprising a non-constitutive promoter operably linked to a polynucleotide encoding a functional plant GAD enzyme. In one embodiment, the cell is a plant cell. The invention also provides plants having incorporated therein a foreign gene comprising a non-constitutive promoter operably linked to a polynucleotide encoding a functional plant GAD enzyme.

In another form of the invention, there is provided a chimeric polynucleotide causing increased GABA production in a plant cell transformed therewith, which includes a regulatory sequence comprising a non-constitutive promoter; and a nucleic-acid fragment encoding a functional plant GAD enzyme. In one embodiment, the nucleic acid fragment comprises a member selected from the group consisting of (i) a nucleic acid fragment encoding an enzyme having the amino acid sequence of SEQ ID NO: 2, 4, 6, 8, 10, 12, 14, 16 or 18; (ii) a nucleic acid fragment encoding an enzyme having an amino acid sequence of SEQ ID NO: 2, 4, 6, 8, 10, 12, 14, 16 or 18, encompassing amino acid substitutions, additions and deletions that do not eliminate the function of the enzyme; (iii) a nucleic acid fragment of SEQ ID NO: 1, 3, 5, 7, 9, 11, 13, 15 or 17; and (iv) a nucleic acid fragment of SEQ ID NO: 1, 3, 5, 7, 9, 11, 13, 15 or 17, encompassing base changes that do not eliminate the function of the encoded enzyme.

In another form, the invention provides a method for making a transformed plant that includes (1) providing a vector comprising a constitutive promoter operably linked to a polynucleotide that encodes a plant GAD enzyme; (2) transforming one or more plants with the vector to provide one or more transformed plants that express the polynucleotide; and (3) selecting a transformed plant that (i) exhibits a GABA concentration in non-stress conditions of up to about 0.20 milligrams GABA per gram dry weight of the plant; or (ii) does not exhibit significant loss of growth characteristics, yield, reproductive function or other morphological or agronomic characteristic compared to a non-transformed plant. In one embodiment, the GAD enzyme is a modified GAD that does not include a functional autoinhibitory calmodulin-binding domain. In another embodiment, the transformed plant produces GAD enzymes at a rate substantially greater than the rate at which GAD enzymes are produced by a non-transformed plant of the same species under the same conditions. The plant can be transformed by (i) transforming a cell, tissue or organ from a host plant with the DNA construct; (ii) selecting a transformed cell, cell callus, somatic embryo, or seed which contains the DNA construct; (iii) regenerating a whole plant from the selected transformed cell, cell callus, somatic embryo, or seed; and (iv) selecting a regenerated whole plant that expresses the polynucleotide. The invention also provides a plant transformed using the method.

In another aspect of the invention, there is provided a plant transformed with a vector comprising a constitutive promoter operably linked to a polynucleotide that encodes a GAD enzyme, or progeny thereof. The plant expresses the polynucleotide; and the plant (i) exhibits a GABA concentration in non-stress conditions of up to about 0.20 milligrams GABA per gram dry weight of the plant; or (ii) does not exhibit significant loss of growth characteristics, yield, reproductive function or other morphological or agronomic characteristic compared to a non-transformed plant. In one embodiment, the plant is selected from the group consisting of duckweed, rice, wheat, barley, rye, corn, Bermuda grass, Blue grass, fescue, rapeseed, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, bush beans, tobacco, tomato, green pepper, sorghum and sugarcane.

Any experiments, experimental examples, or experimental results provided herein are intended to be illustrative of the present invention and should not be considered limiting or restrictive with regard to the invention scope. Further, any theory, mechanism of operation, or finding stated herein is meant to further enhance understanding of the present invention and is not intended to limit the present invention in any way to such theory, mechanism or finding. All publications, patents, and patent applications cited in this specification are herein incorporated by reference as if each individual publication, patent, or patent application were specifically and individually indicated to be incorporated by reference and set forth in its entirety herein. While the invention has been illustrated and described in detail in the drawings and foregoing description, the same is to be considered as illustrative and not restrictive in character, it being understood that only selected embodiments have been shown and described and that all changes, equivalents, and modifications that come within the spirit of the invention described herein or defined by the following claims are desired to be protected.

Reference will now be made to specific examples illustrating the nucleic acid molecules, methods and transformed plants described above. It is to be understood that the examples are provided to illustrate preferred embodiments and that no limitation to the scope of the invention is intended thereby.

### EXAMPLES

### Example One

### Construction of Plants with Altered GAD

A series of transgenic plants, Arabidopsis thaliana (WS ecotype), have been developed using a polymerase chain reaction (PCR)-based cloning strategy. The series of transgenic plants expressing one of the following gene constructs have been developed. One set of plants over-express either a GAD1 or GAD2 gene construct, and have been designated sense GAD1 (senGAD1) or GAD2 (senGAD2), respectively. Another set of plants over-express either GAD1 or GAD2 minus their respective CaM-BDs, which constructs were designated truncated GAD1 (trunGAD1) or GAD2 (trunGAD2), respectively, because they contain stop codons prior to the CaM-BDs. The last set of plants over-express an antisense construct for either GAD1 or GAD2, which constructs were designated antisense GAD1 (antiGAD1) or GAD2 (antiGAD2), respectively.

The polymerase chain reaction (PCR) was used to engineer plants that constitutively expressed one of the above mentioned constructs: senGAD1, senGAD2, trunGAD1, trunGAD2, anitGAD1, or anitGAD2. The engineering strategy for each construct was the same. The nucleotide sequence for each cDNA, either GAD1 or GAD2, was analyzed using MacVector (Oxford Molecular Group, Inc., Beaverton, Oreg.) software to identify restriction enzymes. The nucleic acid sequence, or the recognition site, for a restriction enzyme that was missing from the cDNA sequence but that was present in the plant vector, pPV1 (described below) was added to the 5′-ends of gene specific primers. A pair of gene specific primers was commercially synthesized for the synthesis of the sense and antisense GAD1 and GAD2 constructs. Each of the 5′primers begins with four nucleotides (GCCC) before the nucleotide sequence corresponding to the recognition sequence of the chosen restriction enzyme to increase the efficiency of the restriction digest. The next nucleotides correspond to the first 24 to 29 bases of the open reading frame beginning with the predicted translation initiation site (ATG) for the gene. Similarly the 3′primer begins with four nucleotides (GCCC) before the nucleotide sequence corresponding to the recognition sequence of the chosen restriction enzyme. This is followed by the inverse complement of the last 24 to 29 bases of the open reading frame starting with the predicted translation termination site (TAA, TGA, or TAG). The predicted translation initiation and termination sites were obtained from GenBank submissions. Nucleic acid sequences including the open reading frames of GAD1 and GAD2 were amplified and cloned into the plant expression-vector pPV1 (explained below). Oligonucleotide primers were synthesized with restriction sites for the sequences for the GAD1 and GAD2 constructs are seen in Table 1. In Table 1, all sequences are written 5′ to 3′. Unique restriction sites (Xba I, bold type) were added 5′ to the predicted translation initiation site (ATG), underlined, and predicted inverse complement of the translation termination site (TAA), underlined stop. An additional four nucleotides (GCCC) was added to the 5′-end increase the probability of complete digestion by the restriction enzyme.

TABLE 1

| Primer | |
|---|---|
| Name | Sequence |
| | START |
| | XbaI STOP |
| 5′GAD1 | 5′-GCCCTCTAGAATGGTGCTCTCCCACGCCGTATC-3′ |
| 3′GAD1 | 5′-GCCCTCTAGATTAGCAGATACCACTCGTCTTC-3′ |
| 3′trun- | 5′-GCCCTCTAGATTAGCTCTTCTTCACCGTGACC-3′ |
| GAD1 | |
| | START |
| | XbaI STOP |
| 5′GAD2 | 5′-GCCCTCTAGAATGGTTTTGACAAAAACCGCAA-3′ |
| 3′GAD2 | 5′-GCCCTCTAGATTAGCACACACCATTCATCTTCTT-3′ |
| 3′trun- | 5′-GCCCTCTAGATTACATCTTCTTCTCCTTTACA-3′ |
| GAD2 | |

Separate amplification reactions were conducted with 5′- and 3′-GAD-specific primers with the appropriate cDNA clone as a template using a gene amplification kit (PanVera Corporation, Madison, Wis.). Amplification reactions were conducted as follows: 94° C. for 30 seconds, 55° C. for 30 seconds and 72° C. for 4 minutes, for 25 cycles. The amplified fragments were digested with Hind III, ligated into the vector, and transformed into XL1-Blue MRF′ (Stratagene, La Jolla, Calif.) competent cells. The correct orientation was verified by restriction endonuclease, PCR, or sequence analyses.

For the amplification of the sense and antisense of any GAD construct the same set of 5′- and 3′-primers were used. Since both ends of the DNA had similar restriction sites the insert will theoretically clone in either direction of the appropriately cut vector. The direction of the insert in the vector was determined by one of the following methods, (a) restriction enzyme analysis, (b) PCR with a gene-specific and vector-specific primer or (c) DNA sequence analysis.

Each cassette had unique restriction enzyme sites added to the 5′- and 3′-ends. The amplified fragments were cloned into a PCR cloning vector. The sequence of each construct was confirmed prior to cloning the cassette into the plant transformation vector, pPV1 using standard cloning techniques. The vector, pPV1, is a modified pBI121 (Clonetech) vector minus GUS but with additional unique restriction sites. The vector contains the CaMV 35S promoter and the nopaline synthase terminator. The orientation of the cloned constructs were confirmed by restriction endonuclease and PCR analyses. Upon completion of cloning, the binary vector construct was transferred into a disarmed strain of Agrobacterium tumefaciens, EHA105, and subsequently into Arabidopsis (Ws ecotype) using the vacuum infiltration method (Bechtold, N. and Bouchez, D. (1995) In planta Agrobacterium-mediated transformation of adult Arabidopsis thaliana plants by vacuum infiltration. In Gene Transfer to Plants. I. Potrykus and G. Spangenberg Eds. Springer-Verlag, Heidelberg, pp. 19-23) with one modification, the addition of 0.02% (v/v) Silwet to the infiltration media. Seeds collected from the transformed plants were germinated and selected for kanamycin resistance. Thirteen individual antiGAD2 plants, 13 individual senGAD2, 20 individual trunGAD2 and 15 individual pPV1 plants were kanamycin resistant. These plants have been self-crossed, selected for 100% kanamycin and analysis of the T3 plants has been initiated.

### Example Two

### Rapid Accumulation of GABA in Arabidopsis After Mechanical Stimulation (MS)

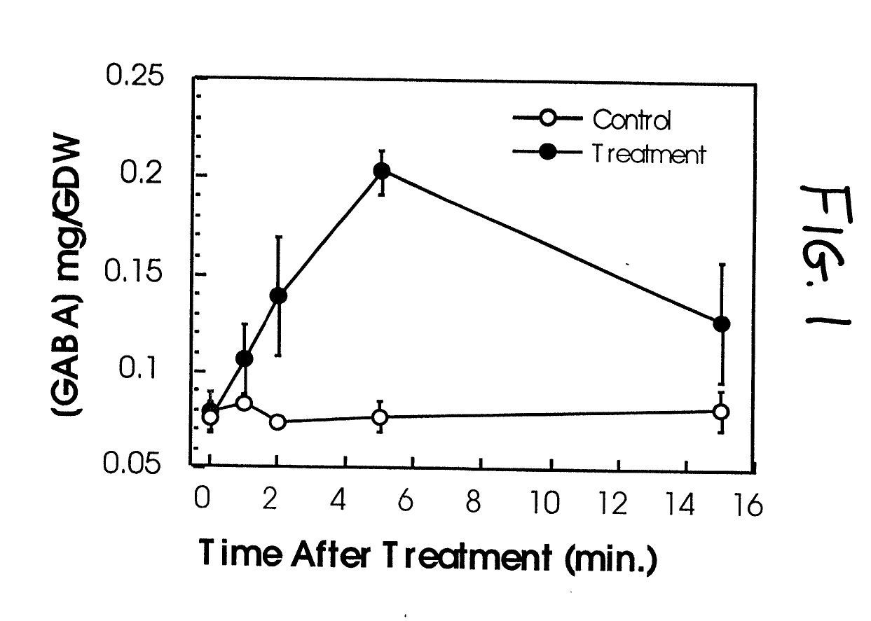

Analysis was conducted of the rapid and transient accumulation of GABA in wild-type Arabidopsis after mechanical stimulation (MS). Plants were individually potted in 2 inch containers and maintained as previously described by Turano and Fang (1998). Two week-old plants were subjected to MS (1 gm/cm²), and plants were harvested at different time intervals (1, 2, 5, 15 minutes) after MS. The samples were immediately frozen in liquid nitrogen and the free amino acids were determined. The values in FIG. 1 are the mean of five samples and the bars represent ±SD.

To determine the amount of free amino acids, frozen samples were ground to a powder in liquid nitrogen. Free amino acids were extracted with 95% ethanol from 100 mg of lyophilized tissue. Internal standards were added to each sample to determine yield and to quantify the amount of each amino acid. Samples were derivatized using N-hydroxysuccinimidyl-6-aminoquinoyl carbamate (AMQ) (Cohen, S. A. and Michaud, D. P. (1992) Highly accurate, high sensitivity amino acid analysis with novel carbamates as pre-column derivating reagents.) using an automated system for mixing and injection (Millipore). Derivatized amino acids were separated by reversed phase chromatography and the peaks were detected fluorometrically.

The results show that there is a rapid and transient elevation of free GABA in the plants after MS. GABA titers peak in five minutes and the titers return to normal fifteen minutes after MS.

### Example Three

### GABA Accumulation in antiGAD2 Arabidopsis After Mechanical Stimulation (MS)

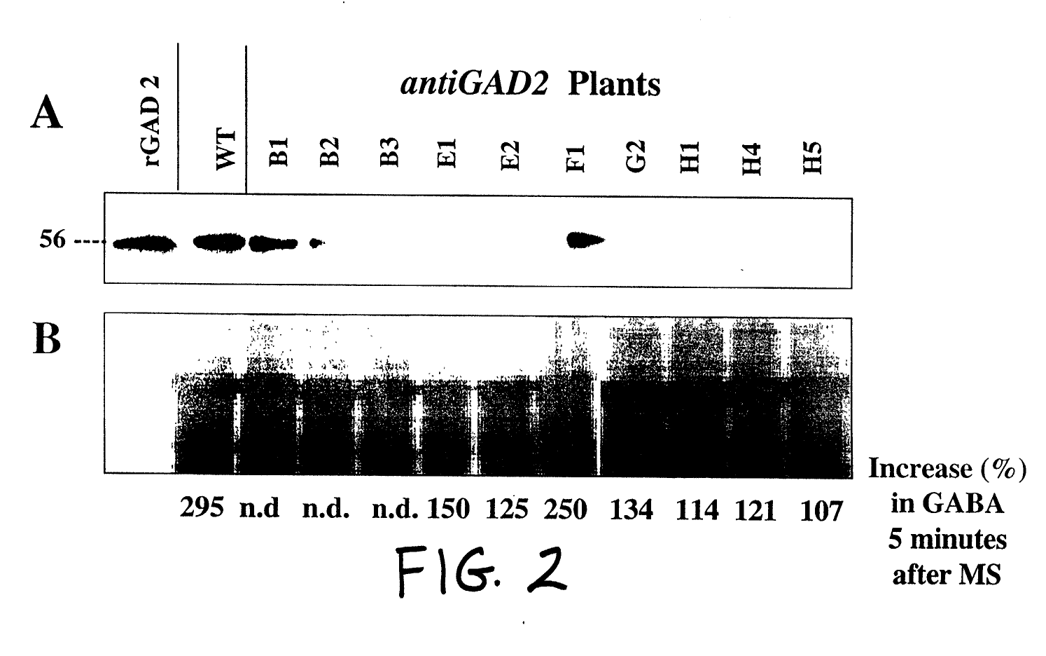

Proteins from kanamycin resistant T3 antiGAD2 plants were extracted from several siblings and immunoblot analysis was used to identify GAD peptides (FIG. 2). Siblings E1 & E2 and H1, H4 & H5 have no detectable amounts of GAD2 peptide and they did not accumulate GABA five minutes after mechanical stimulation (MS). The details of the MS treatment are explained below. The phenotypes of the antiGAD2 plants appear normal when maintained under normal growth conditions, in chambers with little of no air movement and/or vibration at 20-22° C. Due to the low levels of GABA in unperturbed Arabidopsis, it is extremely difficult to assess the relative differences in the accumulation of GABA in unstressed antiGAD and wild-type plants.

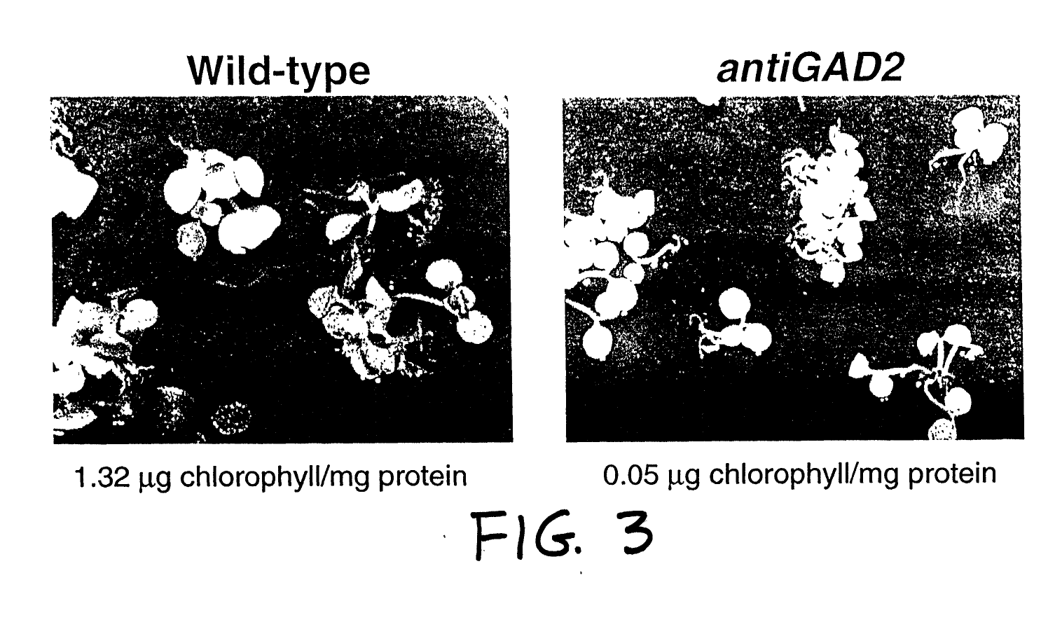

FIG. 2 represents an immunoblot analysis of wild type and antiGAD2 plants. The proteins were loaded equally (75 ug/lane) in wells, separated by SDS-PAGE (8% polyacrylamide), blotted to nitrocellulose, stained for protein to confirm equal loading (B), destained, and GAD2 peptide (A) was detected by immunoblot analysis using a chemiluminescent detection system (SuperSignal, Pierce, Rockville, Ill.). The first lane in FIG. 2A contains recombinant GAD2 (rGAD,56 kDa) as a positive control. See Turano and Fang (1998) for a description of the cloning, expression and purification of rGAD2 from E. coli. The second lane contains protein extracts from a wild-type Arabidopsis. The remaining lanes contain protein extracts from different antiGAD2 plants. The first lane in FIG. 2B contains no sample. The values at the bottom of each lane represent the increase in GABA (%), between a MS and non-MS treated plant, five minutes after MS (1 gm/cm ²). See FIG. 1 for the description of the MS experiment. In FIG. 2 n.d.=not determined. It should be noted that siblings E1 & E2 and H1, H4 & H5 were tested for their response to heat shock, antiGAD2-H1 plants are pictured in FIG. 3. All of the plants were less tolerant to heat shock than wild type or pPV1 plants.

### Example Four

### Evidence that AntiGAD2 Plants do not Tolerate Heat Shock

An initial experiment was performed to test whether GAD2 may play a role in the plant response to heat shock. AntiGAD2, pPV1 (vector control) and wild type plants were grown at normal temperatures (20-21° C.) for two weeks, subjected to heat shock (42° C.) treatment for two hours, and returned to 20-21° C. Twenty-four hours later the antiGAD2 plants did not survive the heat shock treatment, the leaves turned pale green to white (FIG. 3) and the chlorophyll content decreased dramatically. These are the first data to suggest that GAD2 may play a role in plant response to heat shock.

### Example Five

### Evidence that Mechanical Stimulation, GABA Accumulation and Plant Development may be Linked

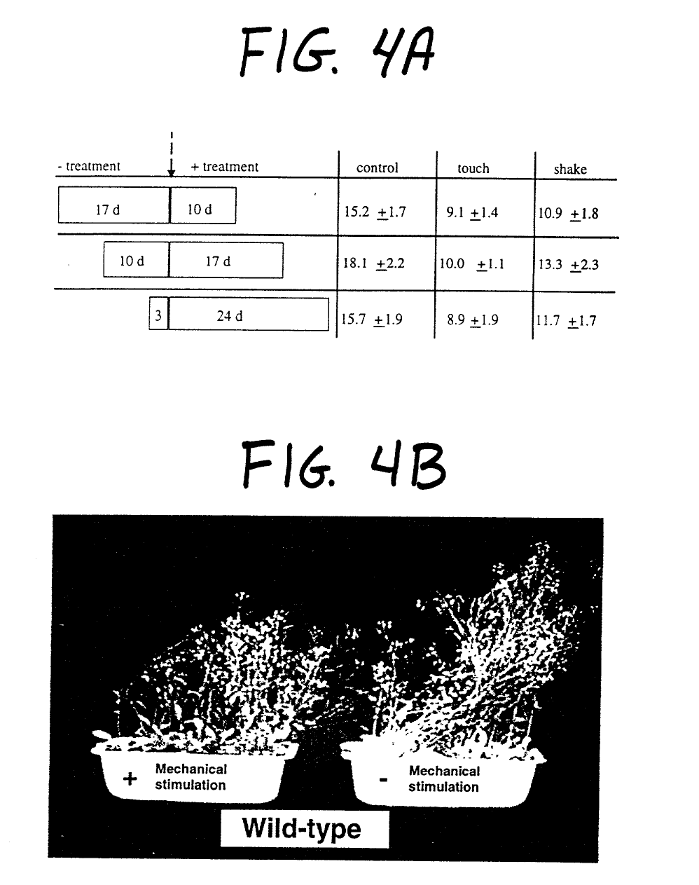

Elevated GABA titers have been reported in plants after MS, and a similar phenomenon has been demonstrated in Arabidopsis (FIG. 1). It has been reported that long-term repeated MS alters plant growth, development, and morphological changes, termed thigmomorphogensis. Similar changes have been observed in Arabidopsis. FIG. 4 demonstrates that repeated MS of Arabidopsis caused morphological changes. The touched plants were 40% shorter than the control plants and had less variability in bolt height than the plants stimulated on a rotor shaker (FIG. 4a). Plants were maintained in chambers with little or no air movement as described by Turano and Fang (1998) for either 3, 10 or 17 days. Plants were mechanically stimulated, either by being touched (1 g/cm²) twice daily or by continuous shaking on a rotor shaker at 100 rpm for a period of 21, 14 or 7 days, respectively. The height of the bolts were recorded on 24 day-old plants (n=25±SD). FIG. 4b demonstrates the difference between touched and nontouched plants.

### Example Six

### Evidence that Overproduction of GABA via Expression of trunGAD Alters Plant Growth

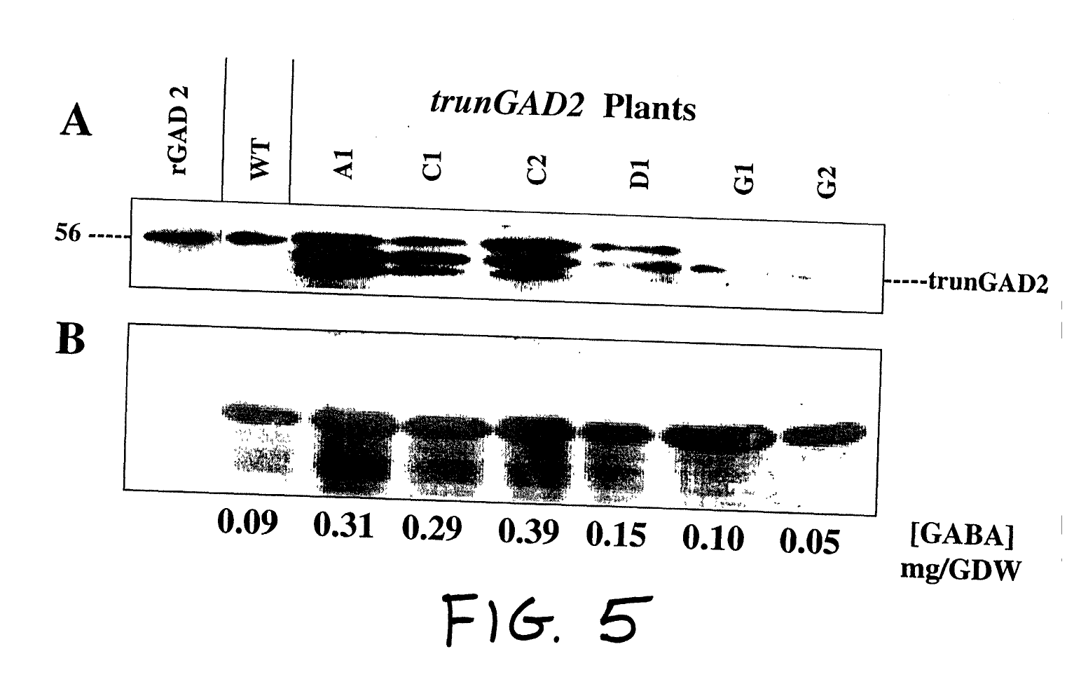

The trunGAD2 plants have been confirmed by immunoblot analysis. Proteins from the kanamycin resistant T3 trunGAD2 plants were extracted from several siblings and immunoblot analysis was used to identify GAD peptides. Both the endogenous GAD2 and trunGAD2 (FIG. 5) are apparent in most samples. Some samples have no native GAD2 peptide but low levels of the trunGAD2 peptide. GAD activity has not been determined in these plants. The numbers under each lane of the gel that was stained for protein in FIG. 5B represent the amount of GABA in mg per gram dry weight (GDW). The first lane in the immunoblot of FIG. 5A contains recombinant GAD2 (rGAD,56 kDa) as a positive control.

Our initial results from the trunGAD2 plants suggest that the plants can be categorized into three groups. One group is similar to those observed by of Baum et al. (Baum, G., Lev-Yadun, S., Fridmann, Y., Arazi, T., Katsnelson, H., Zik, M., and Fromm, H. (1996) Calmodulin binding to glutamate decarboxylase is required for regulation of glutamate and GABA and normal development in plants. EMBO J 15: 2988-2996). They have high GABA and low Glu compared to control plants and are stunted, produce little or no seed (plants A1 and siblings C1 and C2, FIG. 5 and others, data not shown). Another group has moderately higher GABA compared to control plants and the plants are taller than controls and produce viable seed (plant D1, FIG. 5 and others, data not shown). The last group appears normal, with normal or below normal GABA levels but little or no GAD2 peptide and visible amounts of the trunGAD2 (siblings G1 and G2, FIG. 5). We have been able to collect viable seeds from almost all of the trunGAD2 transformed lines, although with very low yields in some. The differences between our results and those of Baum et al. (1996) could be explained by the fact that we have several distinct trunGAD2 transformants (20 individual transformed plants). The variability we observe seems to represent different levels of GAD2 and trunGAD2 due to position effects associated with the gene insertion event. The variability in GAD2 and trunGAD2 is reflected in GABA accumulation and growth and reproductive characteristics of the trunGAD2 plants. Kathiresan et al. (Kathiresan, A., Miranda, J., Chinnappa, C. C. and Reid, D. M. (1998) γ-amino butyric acid promotes stem elongation in Stellaria longipes: the role of ethylene. Plant Growth Reg. 26: 131-136) and Kinnersley and Lin (Kinnersley, A. and Lin, F. (2000) Receptor modifiers indicate that 4-aminobutyric acid (GABA) is a potential modulator of ion transport in plants. Plant Growth Reg. 32:65-76) observed similar relationships between GABA levels and growth characteristics. In both studies low levels of exogenous GABA stimulated cell elongation (Kathiresan et al., 1998) or plant growth (Kinnersley and Lin, 2000) but high concentrations of exogenous GABA inhibited cell elongation and plant growth.

### Example Seven

### Plants Over-Expressing GAD2 Appear Normal

The T3 plants for the senGAD2 and the pPV1 have been partially characterized. The phenotypes of the senGAD2, and pPV1, plants appear normal when the plants are maintained under normal growing conditions. Similar observations were reported for transgenic tobacco plants over-expressing a full-length petunia GAD (Baum et al., 1996).

While the invention has been illustrated and described in detail in the foregoing description, the same is to be considered illustrative and not restricted in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method for making a transformed plant that selectively increases production of GABA in response to a signal, comprising:
incorporating into a plant's genome a DNA construct comprising a non-constitutive promoter operably linked to a polynucleotide that encodes a functional plant GAD enzyme, to provide a transformed plant;
wherein the transformed plant expresses the polynucleotide in response to a signal.

2. The method according to claim 1, wherein the promoter is selected from the group consisting of a tissue preferred promoter, a tissue specific promoter, a cell type specific promoter and an inducible promoter.

3. The method according to claim 2, wherein the promoter is a tissue preferred promoter.

4. The method according to claim 2, wherein the promoter is a tissue specific promoter.

5. The method according to claim 2, wherein the promoter is a cell type specific promoter.

6. The method according to claim 2, wherein the promoter is an inducible promoter.

7. The method according to claim 6, wherein the inducible promoter is responsive to a signal selected from the group consisting of mechanical shock, heat, cold, salt, flooding, drought, wounding, anoxia, pathogens, ultraviolet-B, nutritional deprivation, a flowering signal, a fruiting signal, cell specialization and combinations thereof.

8. The method according to claim 1, wherein the GAD enzyme comprises an amino acid sequence selected from the group consisting of the sequence set forth in SEQ ID NO: 2; the sequence set forth in SEQ ID NO: 4; the sequence set forth in SEQ ID NO: 6; the sequence set forth in SEQ ID NO: 8; the sequence set forth in SEQ ID NO: 10; the sequence set forth in SEQ ID NO: 12; the sequence set forth in SEQ ID NO: 14; the sequence set forth in SEQ ID NO: 16; the sequence set forth in SEQ ID NO: 18 and a sequence having at least about 60% identity thereto that is effective to catalyze a reaction of glutamic acid to GABA.

9. The method according to claim 1, wherein the GAD enzyme is a modified GAD that does not include a functional autoinhibitory calmodulin-binding domain.

10. The method according to claim 1, wherein the transformed plant produces GAD enzymes in response to the signal at a rate greater than the rate at which GAD enzymes are produced by a non-transformed plant of the same species under the same conditions.

11. The method according to claim 1, wherein the target plant is selected from the group consisting of duckweed, rice, wheat, barley, rye, corn, Bermuda grass, Blue grass, fescue, rapeseed, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, bush beans, tobacco, tomato, green pepper, sorghum and sugarcane.

12. The method according to claim 1, wherein the polynucleotide is selected from the group consisting of the nucleotide sequence of SEQ ID NO: 1; the nucleotide sequence of SEQ ID NO: 3; the nucleotide sequence of SEQ ID NO: 5; the nucleotide sequence of SEQ ID NO: 7; the nucleotide sequence of SEQ ID NO: 9; the nucleotide sequence of SEQ ID NO: 11; the nucleotide sequence of SEQ ID NO: 13; the nucleotide sequence of SEQ ID NO: 15; the nucleotide sequence of SEQ ID NO: 17 and sequences that hybridize thereto under moderately stringent conditions.

13. The method of claim 1, wherein said incorporating comprises:
(i) transforming a cell, tissue or organ from a host plant with the DNA construct;
(ii) selecting a transformed cell, cell callus, somatic embryo, or seed which contains the DNA construct;
(iii) regenerating a whole plant from the selected transformed cell, cell callus, somatic embryo, or seed; and
(iv) selecting a regenerated whole plant that expresses the polynucleotide.

14. A transformed plant obtained according to the method of claim 1 or progeny thereof.

15. The transformed plant according to claim 14, wherein the DNA construct is incorporated into the plant in a homozygous state.

16. A DNA construct comprising a non-constitutive promoter operably linked to a polynucleotide that encodes a GAD enzyme; wherein the promoter regulates expression of the polynucleotide in a host cell in response to a signal.

17. The DNA construct in accordance with claim 16, wherein the GAD enzyme comprises an amino acid sequence selected from the group consisting of the sequence set forth in SEQ ID NO: 2; the sequence set forth in SEQ ID NO: 4; the sequence set forth in SEQ ID NO: 6; the sequence set forth in SEQ ID NO: 8; the sequence set forth in SEQ ID NO: 10; the sequence set forth in SEQ ID NO: 12; the sequence set forth in SEQ ID NO: 14; the sequence set forth in SEQ ID NO: 16; the sequence set forth in SEQ ID NO: 18 and a sequence having at least about 60% identity thereto that is effective to catalyze a reaction of glutamic acid to GABA.

18. The DNA construct according to claim 16, wherein the polynucleotide comprises a nucleotide sequence selected from the group consisting of the nucleotide sequence of SEQ ID NO: 1; the nucleotide sequence of SEQ ID NO: 3; the nucleotide sequence of SEQ ID NO: 5; the nucleotide sequence of SEQ ID NO: 7; the nucleotide sequence of SEQ ID NO: 9; the nucleotide sequence of SEQ ID NO: 11; the nucleotide sequence of SEQ ID NO: 13; the nucleotide sequence of SEQ ID NO: 15; the nucleotide sequence of SEQ ID NO: 17 and sequences that hybridize thereto under moderately stringent conditions.

19. The DNA construct according to claim 16, wherein the promoter is a tissue specific plant promoter.

20. The DNA construct according to claim 16, wherein the promoter is an inducible plant promoter.

21. A vector useful for transforming a cell, said vector comprising the DNA construct according to claim 16.

22. A plant transformed with the vector of claim 21, or progeny thereof, wherein the plant expresses the polynucleotide in response to a signal.

23. The plant according to claim 22, the plant being selected from the group consisting of duckweed, rice, wheat, barley, rye, corn, Bermuda grass, Blue grass, fescue, rapeseed, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, bush beans, tobacco, tomato, green pepper, sorghum and sugarcane.

24. A cell having incorporated therein a foreign gene comprising a non-constitutive promoter operably linked to a polynucleotide encoding a functional plant GAD enzyme.

25. The cell according to claim 24, wherein the enzyme comprises an amino acid sequence selected from the group consisting of the sequence set forth in SEQ ID NO: 2; the sequence set forth in SEQ ID NO: 4; the sequence set forth in SEQ ID NO: 6; the sequence set forth in SEQ ID NO: 8; the sequence set forth in SEQ ID NO: 10; the sequence set forth in SEQ ID NO: 12; the sequence set forth in SEQ ID NO: 14; the sequence set forth in SEQ ID NO: 16; the sequence set forth in SEQ ID NO: 18 and a sequence having at least about 60% identity thereto.

26. The cell according to claim 24, wherein the cell is a plant cell.

27. A plant having incorporated therein a foreign gene comprising a non-constitutive promoter operably linked to a polynucleotide encoding a functional plant GAD enzyme.

28. The plant according to claim 27, wherein the enzyme comprises an amino acid sequence selected from the group consisting of the sequence set forth in SEQ ID NO: 2; the sequence set forth in SEQ ID NO: 4; the sequence set forth in SEQ ID NO: 6; the sequence set forth in SEQ ID NO: 8; the sequence set forth in SEQ ID NO: 10; the sequence set forth in SEQ ID NO: 12; the sequence set forth in SEQ ID NO: 14; the sequence set forth in SEQ ID NO: 16; the sequence set forth in SEQ ID NO: 18 and a sequence having at least about 60% identity thereto

29. A chimeric polynucleotide causing increased GABA production in a plant cell transformed with the chimeric polynucleotide, comprising:
a regulatory sequence comprising a non-constitutive promoter; and
a nucleic-acid fragment encoding a functional plant GAD enzyme.

30. The chimeric polynucleotide in accordance with claim 29, wherein the nucleic acid fragment comprises a member selected from the group consisting of:
(i) a nucleic acid fragment encoding an enzyme having the amino acid sequence of SEQ ID NO: 2, 4, 6, 8, 10, 12, 14, 16 or 18;
(ii) a nucleic acid fragment encoding an enzyme having an amino acid sequence of SEQ ID NO: 2, 4, 6, 8, 10, 12, 14, 16 or 18, encompassing amino acid substitutions, additions and deletions that do not eliminate the function of the enzyme;
(iii) a nucleic acid fragment of SEQ ID NO: 1, 3, 5, 7, 9, 11, 13, 15 or 17; and
(iv) a nucleic acid fragment of SEQ ID NO: 1, 3, 5, 7, 9, 11, 13, 15 or 17, encompassing base changes that do not eliminate the function of the encoded enzyme.

31. A method for making a transformed plant, comprising:
providing a vector comprising a constitutive promoter operably linked to a polynucleotide that encodes a plant GAD enzyme;
transforming one or more plants with the vector to provide one or more transformed plants that express the polynucleotide; and
selecting a transformed plant that (i) exhibits a GABA concentration in non-stress conditions of up to about 0.20 milligrams GABA per gram dry weight of the plant; or (ii) does not exhibit significant loss of growth characteristics, yield, reproductive function or other morphological or agronomic characteristic compared to a non-transformed plant.

32. The method according to claim 31, wherein the GAD enzyme comprises an amino acid sequence selected from the group consisting of the sequence set forth in SEQ ID NO: 2; the sequence set forth in SEQ ID NO: 4; the sequence set forth in SEQ ID NO: 6; the sequence set forth in SEQ ID NO: 8; the sequence set forth in SEQ ID NO: 10; the sequence set forth in SEQ ID NO: 12; the sequence set forth in SEQ ID NO: 14; the sequence set forth in SEQ ID NO: 16; the sequence set forth in SEQ ID NO: 18 and a sequence having at least about 60% identity thereto that is effective to catalyze a reaction of glutamic acid to GABA.

33. The method according to claim 31, wherein the GAD enzyme is a modified GAD that does not include a functional autoinhibitory calmodulin-binding domain.

34. The method according to claim 31, wherein the transformed plant produces GAD enzymes at a rate substantially greater than the rate at which GAD enzymes are produced by a non-transformed plant of the same species under the same conditions.

35. The method according to claim 31, wherein the polynucleotide is selected from the group consisting of the nucleotide sequence of SEQ ID NO: 1; the nucleotide sequence of SEQ ID NO: 3; the nucleotide sequence of SEQ ID NO: 5; the nucleotide sequence of SEQ ID NO: 7; the nucleotide sequence of SEQ ID NO: 9; the nucleotide sequence of SEQ ID NO: 11; the nucleotide sequence of SEQ ID NO: 13; the nucleotide sequence of SEQ ID NO: 15; the nucleotide sequence of SEQ ID NO: 17 and sequences that hybridize thereto under moderately stringent conditions.

36. The method of claim 31, wherein said transforming comprises:
(i) transforming a cell, tissue or organ from a host plant with the DNA construct;
(ii) selecting a transformed cell, cell callus, somatic embryo, or seed which contains the DNA construct;
(iii) regenerating a whole plant from the selected transformed cell, cell callus, somatic embryo, or seed; and
(iv) selecting a regenerated whole plant that expresses the polynucleotide.

37. A transformed plant obtained according to the method of claim 31 or progeny thereof.

38. A plant transformed with a vector comprising a constitutive promoter operably linked to a polynucleotide that encodes a GAD enzyme, or progeny thereof;
wherein the plant expresses the polynucleotide; and
wherein the plant (i) exhibits a GABA concentration in non-stress conditions of up to about 0.20 milligrams GABA per gram dry weight of the plant; or (ii) does not exhibit significant loss of growth characteristics, yield, reproductive function or other morphological or agronomic characteristic compared to a non-transformed plant.

39. The plant according to claim 38, the plant being selected from the group consisting of duckweed, rice, wheat, barley, rye, corn, Bermuda grass, Blue grass, fescue, rapeseed, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, bush beans, tobacco, tomato, green pepper, sorghum and sugarcane.

40. A non-sterile plant having incorporated into its genome a foreign DNA construct comprising a promoter operably linked to a polynucleotide that encodes a functional plant GAD enzyme, the polynucleotide comprising a nucleotide sequence selected from the group consisting of the nucleotide sequence of SEQ ID NO: 1; the nucleotide sequence of SEQ ID NO: 3; the nucleotide sequence of SEQ ID NO: 5; the nucleotide sequence of SEQ ID NO: 7; the nucleotide sequence of SEQ ID NO: 9; the nucleotide sequence of SEQ ID NO: 11; the nucleotide sequence of SEQ ID NO: 13; the nucleotide sequence of SEQ ID NO: 15; the nucleotide sequence of SEQ ID NO: 17 and sequences that hybridize thereto under moderately stringent conditions; wherein the plant expresses the polynucleotide.


SEQUENCE LISTING (24 sequences)

SEQ ID NO: 1 - DNA, 1509 bp, Arabidopsis thaliana
  CDS (1)..(1509)
atggtgctctcccacgccgtatcggagtcggacgtctccgtccactccacattcgcatcacgttacgtccgtacttcacttcctaggttcaagatgccggaaaactcgattcctaaggaagcggcgtatcagatcatcaacgacgagctgatgcttgacgggaatccacggttgaacttagcctcctttgtgacgacatggatggagcctgagtgtgataaactcatcatgtcctccatcaacaagaactatgttgacatggacgagtaccccgtcaccaccgaacttcagaaccgatgtgtgaacatgattgcacatctattcaatgcaccgttagaagaggcggagaccgccgtcggagtaggaaccgttggatcatcggaggccataatgttggccggtttggccttcaagcgtaaatggcagaacaagcgcaaagctgaaggcaaacccgtcgataaacccaacattgtcaccggagccaatgttcaagtgtgttgggagaaattcgctaggtactttgaggttgaacttaaggaagtgaaattgagtgaaggatactatgtgatggaccctcaacaagctgttgatatggttgatgagaacaccatttgtgttgcggacattcttggttccactcttaatggagaattcgaagatgttaaactcttgaacgatctcttggtcgaaaagaacaaagaaaccggatgggatacaccaatccacgtggatgcggcaagtggaggattcattgcaccgtttttgtatccggaattggaatgggactttagacttcccttggtgaagagtatcaatgtgagtggtcacaagtatggacttgtgtacgcagggattggttgggtgatctggagaaacaaagaggatttgcctgaggaactcatcttccatatcaattatcttggtgctgaccaacccacctttactctcaatttctccaaaggttcaagtcaagtcattgctcaatactaccaacttatccgattgggccacgagggttacagaaatgtgatggagaattgcagagagaatatgatcgtcctaagggaaggacttgagaagacagaaaggttcaacatcgtctcaaaggacgagggagtgccacttgtcgctttctccttgaaagatagcagctgtcacactgagttcgaaatctccgacatgcttcgcaggtatggatggatagtgccggcctacacaatgcctccaaatgcacaacacatcactgttcttcgtgtggttatcagagaagatttctcgagaacactcgctgagagacttgtgatcgatatagagaaagtgatgcgtgagctcgatgagcttccttcgagagtgattcacaaaatatcacttggacaagagaagagtgaatctaacagcgataacttgatggtcacggtgaagaagagcgatatcgacaagcagagagatatcatcactggctggaagaagtttgtcgccgacaggaagaagacgagtggtatctgctaa

SEQ ID NO: 2 - protein, 502 aa, Arabidopsis thaliana
MVLSHAVSESDVSVHSTFASRYVRTSLPRFKMPENSIPKEAAYQIINDELMLDGNPRLNLASFVTTWMEPECDKLIMSSINKNYVDMDEYPVTTELQNRCVNMIAHLFNAPLEEAETAVGVGTVGSSEAIMLAGLAFKRKWQNKRKAEGKPVDKPNIVTGANVQVCWEKFARYFEVELKEVKLSEGYYVMDPQQAVDMVDENTICVADILGSTLNGEFEDVKLLNDLLVEKNKETGWDTPIHVDAASGGFIAPFLYPELEWDFRLPLVKSINVSGHKYGLVYAGIGWVIWRNKEDLPEELIFHINYLGADQPTFTLNFSKGSSQVIAQYYQLIRLGHEGYRNVMENCRENMIVLREGLEKTERFNIVSKDEGVPLVAFSLKDSSCHTEFEISDMLRRYGWIVPAYTMPPNAQHITVLRVVIREDFSRTLAERLVIDIEKVMRELDELPSRVIHKISLGQEKSESNSDNLMVTVKKSDIDKQRDIITGWKKFVADRKKTSGIC

SEQ ID NO: 3 - DNA, 1665 bp, Arabidopsis thaliana
  CDS (17)..(1498)
ctaaacagaaacaaagatggttttgacaaaaaccgcaacgaatgatgaatctgtctgcaccatgttcggatctcgctatgttcgcactacacttcccaagtatgagattggtgagaattcgataccgaaagacgctgcatatcagatcataaaagatgagctgatgcttgatggtaacccgaggcttaacctagcttcgtttgtgactacatggatggaaccagagtgtgacaaactcatcatggactctatcaacaagaactacgttgatatggatgagtaccctgtcacaactgagctccagaaccgatgtgtaaacattatagctcgactgttcaatgcgccactcgaggaatctgagacggcggtgggagtagggacagttggttcttcagaagccatcatgttagccggattggccttcaaaagaaaatggcagaacaaacgcaaggctgagggtaaaccctatgacaaacccaacattgtcactggagccaatgttcaagtttgctgggagaaattcgctcggtacttcgaggtggagctaaaggaagtaaacctaagtgaaggttactacgtgatggatccagacaaagcagcagaaatggtagacgagaacacaatctgtgtcgcagccatattgggatccacactcaacggtgagttcgaagacgtgaaacgtctcaatgacttgctagtcaagaaaaacgaggagactggttggaacacaccgatccacgtggatgcagcaagtggagggttcatagctccgtttatctatcctgaattagaatgggactttagacttcctttggttaagagtatcaacgtgagtggtcacaagtatggactggtctatgctggtattggttgggtcgtgtggagggcagcagaggatttgcctgaagagcttatctttcatattaattatcttggtgctgatcaacccactttcactctcaatttctccaagggatcgagccaaattattgctcaatactaccagctcattcgtcttggattcgaggggtacaaaaatgtgatggagaattgcatagagaacatggtggttctcaaagaagggatagagaaaacagagcgtttcaacatagtctcaaaggaccaaggagtgccagtcgtagccttctctctcaaggaccatagtttccacaacgagttcgagatctctgagatgctacgtcgttttggctggatcgtcccagcttacactatgcctgccgatgcacagcacatcacggttctgcgtgttgtcatcagggaagatttctcaagaacactcgcggagagacttgttgctgatatttcgaaggtgcttcatgagctagataccttgccttccaagatatctaagaagatgggaatagaagggatcgcggaaaatgtaaaggagaagaagatggagaaggagattctgatggaagttattgttggatggaggaagtttgtgaaggagaggaagaagatgaatggtgtgtgctaagcaagtgtgttgcctttgtgtggaaatgaagaggtacttgcgaggactttgcgtttatcagtttatgtgtttgtatatctatttgatccagttattatggattatatacgcttgaaactcattttaagccattgttattgaacgtttatcaaatactttattat

SEQ ID NO: 4 - protein, 494 aa, Arabidopsis thaliana
MVLTKTATNDESVCTMFGSRYVRTTLPKYEIGENSIPKDAAYQIIKDELMLDGNPRLNLASFVTTWMEPECDKLIMDSINKNYVDMDEYPVTTELQNRCVNIIARLFNAPLEESETAVGVGTVGSSEAIMLAGLAFKRKWQNKRKAEGKPYDKPNIVTGANVQVCWEKFARYFEVELKEVNLSEGYYVMDPDKAAEMVDENTICVAAILGSTLNGEFEDVKRLNDLLVKKNEETGWNTPIHVDAASGGFIAPFIYPELEWDFRLPLVKSINVSGHKYGLVYAGIGWVVWRAAEDLPEELIFHINYLGADQPTFTLNFSKGSSQIIAQYYQLIRLGFEGYKNVMENCIENMVVLKEGIEKTERFNIVSKDQGVPVVAFSLKDHSFHNEFEISEMLRRFGWIVPAYTMPADAQHITVLRVVIREDFSRTLAERLVADISKVLHELDTLPSKISKKMGIEGIAENVKEKKMEKEILMEVIVGWRKFVKERKKMNGVC

SEQ ID NO: 5 - DNA, 2493 bp, Arabidopsis thaliana
  CDS (387)..(794)
atggttttatctaagacagcttccaaatccgatgattcaatccattcaacttttgcttcccgttatgtccgcaactctatctcacggtaagaagttgaaacacaattttattttgtttaatgttttcattggtaactagagttctaaaacttagcctagacgacgatacacagcatcttgattctagattcaatatttattacagaaatatttatttttaatatacgatatagttccagattttaatttttgggtacataagaaagaatactagattctaacgaaattaaccacttgcactgaaagatccgagcataatgtgtgttactatataagaggtattttcttttttaatcttaagctaaatatatcaatttttcatcagattcgaaatacctaagaactcgatccctaaggaagcagcataccaaatcatcaacgacgagctcaagtttgacggtaacccgaggctaaacctggcctcctttgtgaccacttggatggagccagaatgtgacaagctcatgatggaatccatcaacaagaacaacgttgagatggaccaataccctgttaccaccgaccttcagaatcgatgcgttaacatgattgcgcgtctcttcaacgcgcctttaggtgacggtgaagccgccattggtgttggcacggtggggtcatcggaggcagtgatgttggccggactggcctttaagagacagtggcagaacaagcgtaaggccctagggctgccttatgatagacctaatattgtaaccggagccaatattcaggtaaaccaaaacaaaaattgattaaattttaaaccggtttaggtctatgtttacattgactcaatttccggttcaatacaggtttgcttggagaaatttgcaaggtattttgaagtggagcttaaggaagtgaagctgagagaaggatattacgtgatggaccctgacaaagcggttgaaatggtagacgaaaacactatatgcgtcgtggccatcctcggttcgacactaaccggagaattcgaagacgttaagctcctcaacgaccttttagtcgagaaaaacaagaaaaccgggtaattgaatcaaaaccaactaacaaattaattttatatacttttgcctagaaatattacaatttctaacgtgagatatatttgcttagaaatattttattttttgaatgaatataaaacttattaaccaaaacaaaaccatatatgtttacattatatgcttccttgtatcgaatggtgttttaaatactgattaaaaaatgttttgcttaaaaatataacaatttataatgtgagatattcaagcattctaatatcaaaccgataaacaacaacaaactgattattaatttatttaaccggtttggttccggtttaatatatttgtagatgggatacgccgattcacgtggacgcagcgagtggtgggtttattgctcccttcttgtatccggacttggagtgggatttccggttaccgttggttaagagcataaatgtgagtggtcacaaatacggtttggtttacgccggtatcggttgggtcgtatggagaaccaaaaccgatttgcctgatgaacttatcttccatatcaattatcttggagctgatcaacccacatttaccctcaacttctctaaaggtacattaccatatcttatgtaaagtttagatatatttatagattaatgttttgttaattcttgtatattaccagggtcaagtcaagtgattgctcagtactaccagttgattcgtcttggattcgaggtaaataataactcaataaagaaactaaaacgttactaaatccaatcgtatacgtactagtataatatacaagttgttactatactttatgactacaaaagttcaaaaccaagaatgtactaaatacattccataagattaaacgttcctaaattgacaagttttggttttgtagaatagctaataatctttttgtttggtttagggatatcgcaacgtgatggataattgccgcgagaacatgatggtactaagacaaggattagagaaaacgggacgttttaacatcgtctccaaagaaaacggtgttccgttagtggcgttttctctcaaagatagtagccgccacaacgagttcgaggtggccgaaatgcttcgtcgcttcggctggatcgttccggcctacacgatgcctgcggatgcgcaacatgtcacggtccttcgagttgttatccgagaagatttctctcgaaccttagctgagagattggtagccgatttcgagaaggttctacacgagctcgatacgcttcccgcgagggttcacgccaagatggctagtggaaaagttaacggtgttaagaagacgccagaggagacgcaaagagaagtcacggcctactggaagaagtttgtggacactaagactgacaagaacggcgttccgttagtagcaagtattaccaatcaatga

SEQ ID NO: 6 - protein, 500 aa, Arabidopsis thaliana
MVLSKTASKSDDSIHSTFASRYVRNSISRFEIPKNSIPKEAAYQIINDELKFDGNPRLNLASFVTTWMEPECDKLMMESINKNNVEMDQYPVTTDLQNRCVNMIARLFNAPLGDGEAAIGVGTVGSSEAVMLAGLAFKRQWQNKRKALGLPYDRPNIVTGANIQVCLEKFARYFEVELKEVKLREGYYVMDPDKAVEMVDENTICVVAILGSTLTGEFEDVKLLNDLLVEKNKKTGWDTPIHVDAASGGFIAPFLYPDLEWDFRLPLVKSINVSGHKYGLVYAGIGWVVWRTKTDLPDELIFHINYLGADQPTFTLNFSKGSSQVIAQYYQLIRLGFEGYRNVMDNCRENMMVLRQGLEKTGRFNIVSKENGVPLVAFSLKDSSRHNEFEVAEMLRRFGWIVPAYTMPADAQHVTVLRVVIREDFSRTLAERLVADFEKVLHELDTLPARVHAKMASGKVNGVKKTPEETQREVTAYWKKFVDTKTDKNGVPLVASITNQ

SEQ ID NO: 7 - DNA, 2121 bp, Arabidopsis thaliana
  CDS (1)..(87)
atggttttgtctaagacagtttccgaatctgatgtctcaatccattcaacttttgcttctcgttacgtccgcaactctcttccacggtaacaacttgtaacacaaatcttttgtctaatgttttcgtcaacaatagtaacatgtaatgatgtaaaccttggatagttttttttttggccgtggttaatgttgtagatttattatgtgttatatactataaggaaggacatgtttcgttattttaacttaatgtatcatcatttcatcattagattcgaaatgcctgagaactcaatcccaaaagaagcagcttaccaaatcatcaacgacgagctaatgctcgatggtaacccaaggctgaacctagcttccttcgtgaccacatggatggagccagaatgtgacaagctcatgatggagtccatcaacaagaactacgtcgacatggacgagtaccctgtcaccactgagcttcagaaccgatgtgttaacatgatagcacgtctcttcaacgcgccgcttggtgacggtgaagctgccgttggtgttggcaccgtcggatcgtcggaggcgattatgttggccggtttggcttttaagagacaatggcagaataagcgtaaggcccaagggcttccttatgataagcccaatatcgtaaccggtgctaatgtccaggtaaaccaaaacaaaaattgatgaaatattaaccaagacaaaattgaatttatcaatccggttaagttatatgtgtgactcaatttccggttcaatacaggtttgctgggagaaattcgcaaggtatttcgaagtggagcttaaggaagtgaacctaagagaagactattacgtgatggaccctgtaaaggcggtcgaaatggtagacgaaaacacaatttgtgtcgctgccatcctcggttcaacgttaaccggtgaattcgaagacgttaagctcctcaacgacctccttgtcgagaaaaacaagcaaaccgggtaattaaaccaaaccgagaaacaagctaatatcgattgtaatcggtttggagtccggttttaacgttctaaaacacaatttgcagatgggacacgccaatacacgtggacgcagcgagtggtgggtttattgctccgttcttgtatccggagctggagtgggatttccggctaccgttggttaagagtattaatgtgagtggtcacaaatacggtttggtttacgccggtattggttgggttgtatggagaaccaaaaccgatttgcctgatgaacttatcttccatatcaattatcttggcgctgatcaaccaacctttacactcaacttctccaaaggtacattaccataagtccataacatatataactttcaataatatttttggtgtatggaattgttttatagactaaacatttgataatgcttgtataaaccaggttcaagtcaagtgattgctcagtactaccagctgattcgtcttggattcgaggtaaataataactcaaaatagcaatatatttaccaaatggtcaataaagaaactagaatgtattatatttaagttgttacttgttactatactttgaattaaacgttcctaacatgactagttttggtattgtgtaattaataatgtttttcttgtttgatttagggttatcgcaatgtgatggataattgtcgggaaaacatgatggtactaagacaaggattagagaaaacgggacgttttaaaatcgtctccaaagaaaacggtgttccgttagtggcgttttctctcaaagatagtagccgccacaacgagttcgaggtggcccatacactccgtcgcttcggctggatcgttccggcctacacgatgcctgcggatgcgcagcatgtcactgtccttcgagttgttatccgagaagatttctctcgaaccttagccgagagattggtagctgatttcgagaaggttctacacgagctcgatacgcttccggcgagggttcacgccaagatggctaatggaaaagttaacggtgttaagaagacgccagaggagacgcagagagaagtcacggcctactggaagaagttgttggagactaagaagaccaacaagaacacaatttgctaa

SEQ ID NO: 8 - protein, 493 aa, Arabidopsis thaliana
MVLSKTVSESDVSIHSTFASRYVRNSLPRFEMPENSIPKEAAYQIINDELMLDGNPRLNLASFVTTWMEPECDKLMMESINKNYVDMDEYPVTTELQNRCVNMIARLFNAPLGDGEAAVGVGTVGSSEAIMLAGLAFKRQWQNKRKAQGLPYDKPNIVTGANVQVCWEKFARYFEVELKEVNLREDYYVMDPVKAVEMVDENTICVAAILGSTLTGEFEDVKLLNDLLVEKNKQTGWDTPIHVDAASGGFIAPFLYPELEWDFRLPLVKSINVSGHKYGLVYAGIGWVVWRTKTDLPDELIFHINYLGADQPTFTLNFSKGSSQVIAQYYQLIRLGFEGYRNVMDNCRENMMVLRQGLEKTGRFKIVSKENGVPLVAFSLKDSSRHNEFEVAHTLRRFGWIVPAYTMPADAQHVTVLRVVIREDFSRTLAERLVADFEKVLHELDTLPARVHAKMANGKVNGVKKTPEETQREVTAYWKKLLETKKTNKNTIC

SEQ ID NO: 9 - DNA, 1946 bp, Arabidopsis thaliana
  CDS (1)..(87)
atggtactcgcaaccaactctgactccgacgagcatttgcattccacttttgcttctagatatgtccgtgctgttgttcccaggttccagagagttttgcctcattttagtttttttaatcttgtatgctacattgttatatatttaattatttatgtatctgtttgcatatattgaaacaggttcaagatgcctgaccattgcatgcccaaagatgctgcttatcaagtgatcaatgatgagttgatgcttgatggtaatcccaggcttaacctagcctcctttgtcaccacttggatggaacctgagtgtgacaaactcatcatggattctgtcaataagaactatgttgatatggatgaatatcctgtcaccactgagctccaggttcctccttctttcctctcattctctctctcatctactttccactgttttgtcatagactcatacatcttttatctggcttatttttcagaaccggtgtgtaaatatgatagcaaacttgttccatgctcccgttggagaagacgaggctgctattgggtgtggaactgttggttcatctgaggctataatgcttgctggtttggctttcaaaaggaaatggcaacataggagaaaagctcagggtctacctattgataagcctaacattgtcactggagccaatgttcaggtctaaaatatttacttattcttatcctccaaaccatcacatttgctttggatagtgatctgtttctttccaatatcaatacattttcaaactttgtttcatccgctcaggtgtgctgggagaagtttgcaaggtactttgaggtagagctcaaagaggtgaaactaagtgaagactactatgttatggatccagctaaagctgtagagatggtggatgagaataccatctgtgttgcagcaattctaggatccacacttactggagagtttgaggacgttaagcaattgaacgatctcttagctgagaaaaacgcagagacaggatgggaaactcctattcatgttgatgcagccagtggaggattcattgctcctttcctctaccctgatcttgaatgggactttaggcttccatgggtgaagagtattaacgtcagtggtcacaagtatggacttgtgtatgcaggagttggttgggttgtctggagaacaaaagatgatttgccagaggaacttgtcttccacatcaactacttgggagctgatcaacccactttcactctcaacttctcaaaaggtttgtaaaataaaaactggctttatccaatcaaatccatcatcacatttcctttaagaaactcaatgttttcttttgcagggtcgagccaaatcattgctcagtactatcagtttatccgactaggctttgaggtacttgttcccttatctgcattacagtttcattttttcatcttgcttaatctaatgattctttttggaaactggaaaagggatacaagaacataatggaaaactgcatggataacgcaaggaggctaagagaaggaatagagatgacagggaagttcaacattgtgtccaaagatattggcgtgccactagtggcattctctctcaaagacagtagcaagcacacggtgtttgagatcgcagagtctttgagaaaattcgggtggatcataccggcttacactatgcctgcagatgcacagcacattgctgtgctcagagttgtgataagagaagactttagccgaggccttgcagatagactcatcacacatatcattcaggtgctgaaagagattgaagggcttcctagcaggattgcacatcttgctgcggctgcagcggttagtggtgatgatgaagaagttaaagtgaagactgccaagatgtccttggaggatatcactaagtattggaaacgccttgtggaacacaagagaaatattgtctgctaa

SEQ ID NO: 10 - protein, 494 aa, Arabidopsis thaliana
MVLATNSDSDEHLHSTFASRYVRAVVPRFKMPDHCMPKDAAYQVINDELMLDGNPRLNLASFVTTWMEPECDKLIMDSVNKNYVDMDEYPVTTELQNRCVNMIANLFHAPVGEDEAAIGCGTVGSSEAIMLAGLAFKRKWQHRRKAQGLPIDKPNIVTGANVQVCWEKFARYFEVELKEVKLSEDYYVMDPAKAVEMVDENTICVAAILGSTLTGEFEDVKQLNDLLAEKNAETGWETPIHVDAASGGFIAPFLYPDLEWDFRLPWVKSINVSGHKYGLVYAGVGWVVWRTKDDLPEELVFHINYLGADQPTFTLNFSKGSSQIIAQYYQFIRLGFEGYKNIMENCMDNARRLREGIEMTGKFNIVSKDIGVPLVAFSLKDSSKHTVFEIAESLRKFGWIIPAYTMPADAQHIAVLRVVIREDFSRGLADRLITHIIQVLKEIEGLPSRIAHLAAAAAVSGDDEEVKVKTAKMSLEDITKYWKRLVEHKRNIVC

SEQ ID NO: 11 - DNA, 1705 bp, Nicotiana tabacum
  CDS (71)..(1558)
aaaatatctccattttctcccttgttttagtctctgatcttctccgtcgtactaccaccactacgccgccatggttctgtccaagacagcgtcggaaagtgacgtctccatccactccactttcgcttcccgatatgttcgtacttctcttccgaggtttaagatgccagagaattcgataccaaaggaagcagcatatcaaatcataaatgatgagcttatgttagatggaaatccaagactaaatttagcatcttttgtgacaacatggatggaaccagagtgtaacaaactgatgatggattccattaacaagaattacgttgacatggatgaataccctgtaaccactgaacttcagaatcgatgtgtaaacatgatagctcatttgtttaacgcaccacttggagatggagagactgcagttggagttggaactgttggatcctctgaggctattatgcttgctggattagctttcaagagaaaatggcaaaataaaatgaaagcccaaggcaagccctgtgacaagcccaatattgtcactggtgccaatgtccaggtgtgttgggagaaatttgcaaggtattttgaagtggagctaaaggaagtaaagttgagtgatggatactatgtgatggaccctgagaaagctgtggaaatggtggatgagaacacaatttgtgtagctgctatcttgggttccacactcaatggtgaatttgaagatgttaagcgcttgaatgacctcttgattgagaagaacaaagaaaccgggtgggacactccaattcatgtggatgcagcaagtggtggatttattgcaccattcctttatccagagcttgaatgggactttagattgccattggtgaagagtataaacgtgagtggtcacaaatatggtcttgtttatgctggtattggttgggccatttggaggaataaggaagacttacctgacgaacttatcttccacattaattatcttggtgctgatcaacctactttcactctcaacttctctaaaggttctagccaagtaattgctcaatattaccaacttattcgcttgggttttgagggttacaagaatgttatggagaattgtcaagaaaatgcaagggtactaagagaaggacttgaaaaaagtggaagattcaacataatatccaaagaaattggagttccattagtagctttctctcttaaagacaacagtcaacacaatgagttcgaaatttctgaaactcttagaagatttggatggattattcctgcatatactatgccaccaaatgctcaacatgtcacagttctcagagttgtcattagagaagatttctcccgtacactcgccgagcgactggtaatagacattgaaaaagtcctccacgagctagacacacttccggcgagggtcaacgctaagctagccgtggccgaggcgaatggcagcggcgtgcataagaaaacagatagagaagtgcagcttgagattactactgcatggaagaaatttgttgctgataagaagaagaagactaacggagtttgttaatttaatttaacaaaatatgtttataattaatatgatgatttataactactagcagtggtactgcttgtttttatatttgaattgttgggttttttgagtatgaggagctagctatttattgctagtgaaatattggttgaaaaa

SEQ ID NO: 12 - protein, 496 aa, Nicotiana tabacum
MVLSKTASESDVSIHSTFASRYVRTSLPRFKMPENSIPKEAAYQIINDELMLDGNPRLNLASFVTTWMEPECNKLMMDSINKNYVDMDEYPVTTELQNRCVNMIAHLFNAPLGDGETAVGVGTVGSSEAIMLAGLAFKRKWQNKMKAQGKPCDKPNIVTGANVQVCWEKFARYFEVELKEVKLSDGYYVMDPEKAVEMVDENTICVAAILGSTLNGEFEDVKRLNDLLIEKNKETGWDTPIHVDAASGGFIAPFLYPELEWDFRLPLVKSINVSGHKYGLVYAGIGWAIWRNKEDLPDELIFHINYLGADQPTFTLNFSKGSSQVIAQYYQLIRLGFEGYKNVMENCQENARVLREGLEKSGRFNIISKEIGVPLVAFSLKDNSQHNEFEISETLRRFGWIIPAYTMPPNAQHVTVLRVVIREDFSRTLAERLVIDIEKVLHELDTLPARVNAKLAVAEANGSGVHKKTDREVQLEITTAWKKFVADKKKKTNGVC

SEQ ID NO: 13 - DNA, 1771 bp, Nicotiana tabacum
  CDS (67)..(1554)
tattttcattttctctcctgttttaatttctgatcttctccgtcgtactaccaccactacgccgccatggttctgtccaagacagcgtcggaaagtgacgtctccgttcactccactttcgcctcccgatatgttcgaacttctcttcccaggtttaaaatgccagagaattcaataccaaaggaagcagcatatcagattataaatgatgagcttatgttagatggaaatccaaggctaaatttagcatctttcgttacaacatggatggagccagaatgtaatacgttaatgatggattccattaacaagaactacgttgacatggatgaataccctgtaaccactgagcttcagaatcgatgtgtaaatatgatagctcatttgtttaatgcaccacttggagatggagagactgcagttggagttggaactgttggatcctctgaagctattatgcttgctggattagcctttaagagaaaatggcaaaataaaatgaaagcccaaggcaagccctttgataagcccaatattgtcaccggtgctaatgtccaggtgtgttgggagaaatttgcaaggtattttgaagtggagttgaaagaagtaaaattgagtgatggatactatgtgatggaccctgagaaagctgtggaaatggtggatgagaataccatttgtgttgctgctatcttaggttcaacactcaatggtgaatttgaagatgttaagcgtttgaatgaccttttgattgagaagaacaaagaaaccgggtgggacactccaattcatgtggatgcagcaagtggtggatttattgcaccattcctttatccagagcttgaatgggactttagattgccattggagaagagtattaatgtgagtggtcacaaatatggtcttgtctatgctggtattggttgggccatttggaggaataaggaagacttgcctgatgaacttattttccacatcaattaccttggtgctgatcaacctactttcactctcaacttctctaaaggttctagccaagtaattgctcaatattaccaacttattcgcttgggttttgagggttacaagaatgttatggagaattgtcaagaaaatgcaagggtattaagagaaggaattgaaaaaagtggaagattcaacataatctccaaagaaattggagttcccttagtagcattttctcttaaagacaacagtcaacacaatgagttcgaaatttctgaaactcttagaagatttggatggattgttctggcatatactatgccaccaaatgctcaacatgtcacagttctcagagttgtcattagagaagatttctcccgcacactagcggagcgactggtaatagacattgaaaaagtcttccacggagtagacacacttccggcgagggtcaacgctaagctagccgtggccgaggcgaatggcagcggcgtgcataagaaaacagatagagaagtgcagctagagattactactgcatggttgaaatttgttgctgataagaagaagaagactaatggagtttgttaatttaatttaacaaaaaaaaagtttataatatggtgatttatgtaactactagcagtcgtactgcttgttttttatatttgagttgatgtgttttttgagcacttgaggagctagctagttattgctagtgaaaaattggatgatatattttggactactttgtaagtttgtattattaatccaaattaaacgatatttatcataaaaaaaaaaa

SEQ ID NO: 14 - protein, 496 aa, Nicotiana tabacum
MVLSKTASESDVSVHSTFASRYVRTSLPRFKMPENSIPKEAAYQIINDELMLDGNPRLNLASFVTTWMEPECNTLMMDSINKNYVDMDEYPVTTELQNRCVNMIAHLFNAPLGDGETAVGVGTVGSSEAIMLAGLAFKRKWQNKMKAQGKPFDKPNIVTGANVQVCWEKFARYFEVELKEVKLSDGYYVMDPEKAVEMVDENTICVAAILGSTLNGEFEDVKRLNDLLIEKNKETGWDTPIHVDAASGGFIAPFLYPELEWDFRLPLEKSINVSGHKYGLVYAGIGWAIWRNKEDLPDELIFHINYLGADQPTFTLNFSKGSSQVIAQYYQLIRLGFEGYKNVMENCQENARVLREGIEKSGRFNIISKEIGVPLVAFSLKDNSQHNEFEISETLRRFGWIVLAYTMPPNAQHVTVLRVVIREDFSRTLAERLVIDIEKVFHGVDTLPARVNAKLAVAEANGSGVHKKTDREVQLEITTAWLKFVADKKKKTNGVC

SEQ ID NO: 15 - DNA, 1785 bp, Petunia x hybrida
  CDS (72)..(1571)
aaagagtacaaactaatatccacttaaattgtatttctccattttctctctttatttagtctgtcataacaatggttctatcaaagacagtgtcgcagagcgatgtgtccattcactccacgtttgcttctcgatatgttcgaacttctcttcccaggtttaaaatgccagataattcgataccaaaagaagcagcatatcagatcataaatgatgaactgatgttagatggaaacccaaggctgaacttggcttcttttgttacaacatggatggaaccagagtgtgataagttgatgatggactctattaacaagaactatgttgatatggatgaatatcctgttaccactgagcttcagaatcgatgtgtaaacatgatagctcatttgtttaatgcaccacttgaagatggagaaactgcagttggagttggaactgttggatcctctgaagccattatgcttgctggattagctttcaagagaaaatggcagaacaaaatgaaagcccaaggcaaaccctgtgacaagcccaacattgttactggtgcaaatgtccaggtgtgctgggagaaatttgcaaggtattttgaagtggagctaaaggaagtaaagcttagtgaaggatactatgtgatggaccctgagaaagctgtggagatggtggatgaaaacaccatttgtgtagctgctatcttaggttccaccctcaatggagaatttgaagacgttaagcgcttgaatgatctcttggtcgagaagaacaaagaaaccgggtgggacactccaattcatgtggatgcagcaagtggtggatttattgcaccgttcatttacccagagcttgagtgggactttagattgccattagtgaagagcattaatgtaagtggtcacaaatatggtcttgtctatgctggtattggttgggtcgtttggaggaacaaggatgatttgcctgatgaacttatcttccacattaattatcttggtgctgatcaacctactttcactctcaacttttctaaaggttctagccaagtaattgctcaatattaccaacttattcgcttgggttatgagggttacaagaatgtgatggagaattgtcaagaaaatgcatcggtactaagagaagggctagaaaagacaggaagattcaacataatctccaaagaaattggagtacctttagtagcattctctcttaaagacaacaggcaacacaacgagttcgagatttctgaaactttaaggagatttggttggattgttcctgcatatactatgccaccaaacgcacaacacattacagttctcagagttgtgatcagagaagatttctcccgtacgcttgcagaacgactggtaagagacatcgaaaaagtccttcatgaacttgacacactccctgcacgtgtcaatgctaagctcgctgtggccgaggagcaggcggctgcgaatggcagcgaggtgcataagaaaacagatagcgaagtgcagttggagatgataactgcatggaagaagtttgttgaagaaaagaagaagaagactaatcgagtttgttaattaattatattagtgtttataatatgatgaatatggctattatcattggtgactgcttgttagtatattagctgtgattatcaccaatatgagtttggttttcttgatttggttcttttcagtacttgaaaagttgttattgatattgtaaaattgtactttttaactatttggattattaatgccaattttctagtgtacttaataaaaa

SEQ ID NO: 16 - protein, 500 aa, Petunia x hybrida
MVLSKTVSQSDVSIHSTFASRYVRTSLPRFKMPDNSIPKEAAYQIINDELMLDGNPRLNLASFVTTWMEPECDKLMMDSINKNYVDMDEYPVTTELQNRCVNMIAHLFNAPLEDGETAVGVGTVGSSEAIMLAGLAFKRKWQNKMKAQGKPCDKPNIVTGANVQVCWEKFARYFEVELKEVKLSEGYYVMDPEKAVEMVDENTICVAAILGSTLNGEFEDVKRLNDLLVEKNKETGWDTPIHVDAASGGFIAPFIYPELEWDFRLPLVKSINVSGHKYGLVYAGIGWVVWRNKDDLPDELIFHINYLGADQPTFTLNFSKGSSQVIAQYYQLIRLGYEGYKNVMENCQENASVLREGLEKTGRFNIISKEIGVPLVAFSLKDNRQHNEFEISETLRRFGWIVPAYTMPPNAQHITVLRVVIREDFSRTLAERLVRDIEKVLHELDTLPARVNAKLAVAEEQAAANGSEVHKKTDSEVQLEMITAWKKFVEEKKKKTNRVC

SEQ ID NO: 17 - DNA, 1783 bp, Lycopersicon esculentum
  CDS (6)..(1511)
aaaaaatggtgttaacaacgacgtcgataagagattcagaagagagcttgcactgtacatttgcatcaagatatgtacaggaacctttacctaagttcaaaatgcctaaaaaatccatgccgaaagaagcagcttatcagattgtaaacgacgagcttatgttggatggtaaccccaggttgaatttagcttcctttgttagcacatggatggagcccgagtgcgataagctcatcatgtcatccattaataaaaactatgtcgacatggatgagtatcctgtcaccactgaacttcaaaatagatgtgttaacatgttagcacatcttttccatgccccggttggtgatgatgagactgcagttggagttggtacagtgggttcatcagaggcaataatgcttgctggccttgctttcaaacgcaaatggcaatcgaaaagaaaagcagaaggcaaacctttcgataagcctaatatagtcactggagctaatgtgcaggtctgctgggaaaaatttgcaaggtattttgaggttgagttgaaggaggtgaaactaaaagaaggatactatgtaatggaccctgccaaagcagtagagatagtggatgagaatacaatatgtgttgctgcaatccttggttctactctgactggggagtttgaggatgtgaagctcctaaacgagctccttacaaaaaagaacaaggaaaccggatgggagacaccgattcatgtcgatgctgcgagtggaggatttattgctcctttcctctggccagatcttgaatgggatttccgtttgcctcttgtgaaaagtataaatgtcagcggtcacaagtatggccttgtatatgctggtgtcggttgggtgatatggcggagcaaggaagacttgcccgatgaactcgtctttcatataaactaccttgggtctgatcagcctacttttactctcaacttctctaaaggttcctatcaaataattgcacagtattatcagttaataagacttggctttgagggttataagaacgtcatgaagaattgcttatcaaacgcaaaagtactaacagagggaatcacaaaaatggggcggttcgatattgtctctaaggatgtgggtgttcctgttgtagcattttctctcagggacagcagcaaatatacggtatttgaagtatctgagcatctcagaagatttggatggatcgtccctgcatacacaatgccaccggatgctgaacacattgctgtactgcgggttgtcattagagaggatttcagccacagcctagctgagagacttgtttctgacattgagaaaattctgtcagagttggacacacagcctcctcgtttgcccaccaaagctgtccgtgtcactgctgaggaagtgcgtgatgacaagggtgatgggcttcatcattttcacatggatactgtagagactcagaaagacattatcaaacattggaggaaaatcgcagggaagaagaccagcggagtctgctaggtctggccacacttgttatctgggctccgcttccatcgccatcctgtagtatgtattacgtgtgttgtttccatcttatgtagtagttggtactgtaatctgtgtaaatgctttcatgatcttggctctgtatatgctaaataagcactgcatttcaagttcctggaagtatttatgtatgaatcaatccgggcataattggtagaatgccctctctgcgtcatctttgaatttcacgtgcaataatatttgaaatctacacctattat

SEQ ID NO: 18 - protein, 502 aa, Lycopersicon esculentum
MVLTTTSIRDSEESLHCTFASRYVQEPLPKFKMPKKSMPKEAAYQIVNDELMLDGNPRLNLASFVSTWMEPECDKLIMSSINKNYVDMDEYPVTTELQNRCVNMLAHLFHAPVGDDETAVGVGTVGSSEAIMLAGLAFKRKWQSKRKAEGKPFDKPNIVTGANVQVCWEKFARYFEVELKEVKLKEGYYVMDPAKAVEIVDENTICVAAILGSTLTGEFEDVKLLNELLTKKNKETGWETPIHVDAASGGFIAPFLWPDLEWDFRLPLVKSINVSGHKYGLVYAGVGWVIWRSKEDLPDELVFHINYLGSDQPTFTLNFSKGSYQIIAQYYQLIRLGFEGYKNVMKNCLSNAKVLTEGITKMGRFDIVSKDVGVPVVAFSLRDSSKYTVFEVSEHLRRFGWIVPAYTMPPDAEHIAVLRVVIREDFSHSLAERLVSDIEKILSELDTQPPRLPTKAVRVTAEEVRDDKGDGLHHFHMDTVETQKDIIKHWRKIAGKKTSGVC

SEQ ID NO: 19 - DNA, 33 bp, Artificial Sequence
  Synthetic oligonucleotide primer
gccctctagaatggtgctctcccacgccgtatc

SEQ ID NO: 20 - DNA, 32 bp, Artificial Sequence
  Synthetic oligonucleotide primer
gccctctagattagcagataccactcgtcttc

SEQ ID NO: 21 - DNA, 32 bp, Artificial Sequence
  Synthetic oligonucleotide primer
gccctctagattagctcttcttcaccgtgacc

SEQ ID NO: 22 - DNA, 32 bp, Artificial Sequence
  Synthetic oligonucleotide primer
gccctctagaatggttttgacaaaaaccgcaa

SEQ ID NO: 23 - DNA, 34 bp, Artificial Sequence
  Synthetic oligonucleotide primer
gccctctagattagcacacaccattcatcttctt

SEQ ID NO: 24 - DNA, 32 bp, Artificial Sequence
  Synthetic oligonucleotide primer
gccctctagattacatcttcttctcctttaca